(19) United States
(12) Patent Application Publication
(10) Pub. No.: US 2021/0030022 A1
(43) Pub. Date: Feb. 4, 2021

(54) NOVEL USE OF SUBSTITUTED CHROMAN-6-OLS

(71) Applicant: DSM IP ASSETS B.V., Heerlen (NL)

(72) Inventors: Laure CLASADONTE, Kaiseraugst (CH); André DUESTERLOH, Kaiseraugst (CH); Weerasinghe INDRASENA, Kaiseraugst (CH); Thomas NETSCHER, Kaiseraugst (CH); René STEMMLER, Kaiseraugst (CH)

(21) Appl. No.: 17/041,861

(22) PCT Filed: Mar. 29, 2019
(86) PCT No.: PCT/EP2019/058058

§ 371 Date: Sep. 25, 2020

(30) Foreign Application Priority Data
Mar. 29, 2018 (EP) 18164852.8

(51) Int. Cl. A23K 20/121; C07D 311/20; A23K 30/00; C07D 311/58; A23K 20/158; A23K 50/40; C07D 311/70; A23K 20/147; A23K 50/80; C07D 311/22; A23K 20/111; A23K 50/75

(52) U.S. Cl. CPC A23K 20/121; C07D 311/20; A23K 30/00; C07D 311/58; A23K 20/158; A23K 50/75; C07D 311/70; A23K 20/147; A23K 50/80; C07D 311/22; A23K 20/111; A23K 50/40

(57) ABSTRACT

The present invention is directed towards the use of substituted chroman-6-ols of formula (I) wherein R¹ and R² are independently from each other H or C₁₋₁₁-alkyl or (CH₂)ₙ—OH with n being an integer from 1 to 4, or R¹ and R² represent together a keto group, 10 A is CHR³ or C(═O), and wherein R³, R⁴ and R⁶ are independently from each other H or C₁₋₄-alkyl, and wherein R⁵ is H or OH or C₁₋₄-alkyl or C₁₋₄-alkoxy, as antioxidants, especially in feed such as pet food and feed ingredients such as fish meal, insect meal and poultry meal, as well as PUFA-containing oil such as marine oil, microbial oil, fungal oil, algal oil and PUFA-containing plant oil. The present invention is further directed towards feed ingredients and feed for insects, aquatic and terrestrial animals comprising such substituted chroman-6-ols of formula (I).

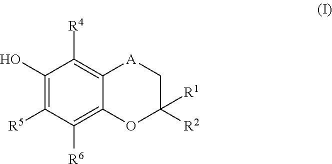

The present invention is directed to the use of a compound of formula (I) as antioxidant,

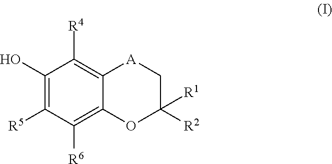

wherein R¹ and R² are independently from each other H or C₁₋₁₁-alkyl or (CH₂)ₙ—OH with n being an integer from 1 to 4, or R¹ and R² represent together a keto group,

### A is CHR³ or C(═O), and

wherein R³, R⁴ and R⁶ are independently from each other H or C₁₋₄-alkyl, and
wherein R⁵ is H or OH or C₁₋₄-alkyl or C₁₋₄-alkoxy.

The compounds of the present invention are efficient as antioxidants, preferably in feed and feed ingredients. The compounds of the present invention are especially efficient as antioxidants in feed comprising proteins and/or unsaturated fatty acid (derivative)s and in feed ingredients comprising proteins and/or unsaturated fatty acid (derivative)s. “Derivatives” are e.g. the monoglycerides, diglycerides and triglycerides as well as C₁₋₆-alkyl esters such as the methyl and ethyl esters.

### BACKGROUND OF THE INVENTION

Unmodified fish meal can spontaneously combust from heat generated by oxidation of the polyunsaturated fatty acids in the fish meal. In the past, factory ships have sunk because of such fires. Strict rules regarding the safe transport of fish meal have been put in place by authorities and the International Maritime Organization (IMO). According to IMO, fishmeal must be stabilized with antioxidants to prevent spontaneous combustion during overseas transport and storage.

The shipping regulations of the United Nations for the Transport of Dangerous Goods (UN-TDG) currently only allow ethoxyquin and BHT as antioxidants to stabilize fish meal for marine transport. But authorization of ethoxyquin has now been suspended in the European Union due to safety and health concerns.

BHT must be added in higher quantities to achieve the same efficacy as ethoxyquin. Furthermore, BHT is currently under safety evaluation by ECHA and its re-registration as feed additive is pending in Europe.

Therefore, there is a need to replace ethoxyquin and BHT as an antioxidant.

### DETAILED DESCRIPTION OF THE INVENTION

This need is fulfilled by the present invention, which is directed to the use of a compound of formula (I) as antioxidant,

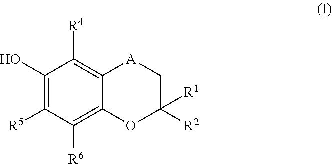

wherein R¹ and R² are independently from each other H or C₁₋₁₁-alkyl or (CH₂)ₙ—OH with n being an integer from 1 to 4, or R¹ and R² represent together a keto group,

### A is CHR³ or C(═O), and

wherein R³, R⁴ and R⁶ are independently from each other H or C₁₋₄-alkyl, and
wherein R⁵ is H or OH or C₁₋₄-alkyl or C₁₋₄-alkoxy;
and with the preferences for the substituents R¹ to R⁶ as given below. “alkyl” and “alkoxy” in the context of the present invention encompass linear alkyl and branched alkyl, and linear alkoxy and branched alkoxy, respectively.

In a preferred embodiment of the present invention R¹ and R² are independently from each other H or C₁₋₁₁-alkyl or (CH₂)ₙ—OH with n being an integer from 1 to 4, A is CHR³, and R³, R⁴ and R⁶ are independently from each other H or C₁₋₄-alkyl and R⁵ is H or C₁₋₄-alkyl or C₁₋₄-alkoxy in the compound of formula (I).

In a further preferred embodiment of the present invention R¹ and R² are independently from each other H or C₁₋₁₁-alkyl or (CH₂)ₙ—OH with n being an integer from 1 to 4, A is CHR³, and R³, R⁴ and R⁶ are independently from each other H or C₁₋₄-alkyl and R⁵ is H or C₁₋₄-alkyl or C₁₋₄-alkoxy in the compound of formula (I) with the proviso that at least one of the substituents R⁴, R⁵ and R⁶ is not methyl.

If one of the substituents R¹ and R² is an C₅₋₁₁-alkyl or if one of R¹ and R² is a (CH₂)ₙ—OH group with 4 C-atoms, the other substituent is preferably H.

More preferably R¹ and R² are independently from each other H or C₁₋₄-alkyl or (CH₂)ₙ—OH with n being 1 or 2, R³, R⁴ and R⁶ are independently from each other H or C₁₋₂-alkyl, and R⁵ is H or C₁₋₂-alkyl or C₁₋₂-alkoxy.

Even more preferably R¹ and R² are independently from each other H or C₁₋₄-alkyl or (CH₂)ₙ—OH with n being 1 or 2, R³, R⁴ and R⁶ are independently from each other H or C₁₋₂-alkyl, and R⁵ is H or C₁₋₂-alkyl or C₁₋₂-alkoxy with the proviso that at least one of the substituents R⁴, R⁵ and R⁶ is not methyl.

Further, more preferably R¹ and R² are independently from each other H or C₁₋₂-alkyl or (CH₂)ₙ—OH with n being 1 or 2, R³, R⁴ and R⁶ are independently from each other H or C₁₋₂-alkyl, and R⁵ is H or C₁₋₂-alkyl or C₁₋₂-alkoxy, preferably with the proviso that at least one of the substituents R⁴, R⁵ and R⁶ is not methyl.

Furthermore, more preferably R¹ and R² are independently from each other H or methyl or (CH₂)—OH, R³, R⁴ and R⁶ are independently from each other H or methyl, and R⁵ is H or methyl or methoxy, preferably with the proviso that at least one of the substituents R⁴, R⁵ and R⁶ is not methyl.

Most preferably R³ is H, preferably with the proviso that at least one of the substituents R⁴, R⁵ and R⁶ is not methyl, more preferably with the proviso that R⁵ and R⁶ are not methyl.

The compound of formula (I) is preferably selected from the group of the compounds of formulae (II) and (III), more preferably from the group of the compounds of formula (IV):

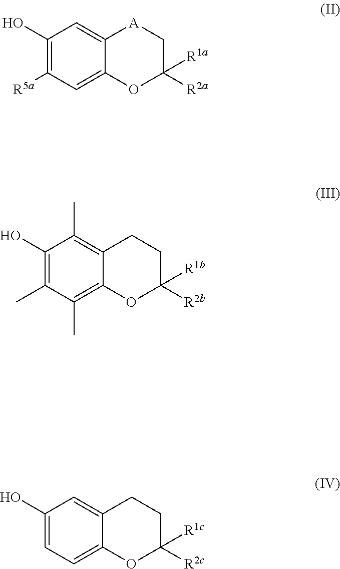

whereby A is CH₂ or C(═O), preferably whereby A is CH₂;
whereby R^{5a} is H or methoxy, preferably whereby R^{5a} is H;
whereby R^{1a} and R^{2a} are independently from each other H, CH₂OH or linear C₁₋₃-alkyl or branched C₄₋₁₁-alkyl or R^{1a} and R^{2a} represent together a keto group (i.e. R^{1a} and R^{2a} are together “═O”), preferably whereby R^{1a} and R^{2a} are independently from each other H, methyl, CH₂OH or [CH₂—CH₂—CH₂—CH(CH₃)]ₘCH₃ with m being 1 or 2 or R^{1a} and R^{2a} represent together a keto group;
whereby R^{1b} and R^{2b} are independently from each other CH₂OH or linear C₁₋₃-alkyl or branched C₄₋₁₁-alkyl, preferably whereby one of Rib and R^{2b} is methyl and the other one of Rib and R^{2b} is CH₂OH or linear C₁₋₃-alkyl or branched C₄₋₁₁-alkyl, more preferably whereby one of R^{1b} and R^{2b} is methyl and the other one of R^{1b} and R^{2b} is methyl, CH₂OH or [CH₂—CH₂—CH₂—CH(CH₃)]ₘCH₃ with m being 1 or 2;
whereby R^{1c} and R^{2c} are independently from each other H or linear C₁₋₃-alkyl or branched C₄₋₁₁-alkyl, preferably whereby R^{1c} and R^{2c} are independently from each other H, methyl or [CH₂—CH₂—CH₂—CH(CH₃)]ₘCH₃ with m being 1 or 2.

Preferred examples of the compound of formula (II) are the compounds of formulae (1), (2), (3), (4), (7), (8), (10) and (11).

Preferred examples of the compound of formula (III) are the compounds of formulae (5), (6) and (9).

Preferred examples of the compound of formula (IV) are the compounds of formulae (1), (2), (3), (7) and (8).

Especially preferred are the following compounds of formulae (1) to (11), whereby compounds of formulae (1) to (8) are more preferred, compounds of formulae (1) to (6) are even more preferred, compounds of formulae (1) to (4) are furthermore preferred, and the most preferred compound is the compound of formula (3):

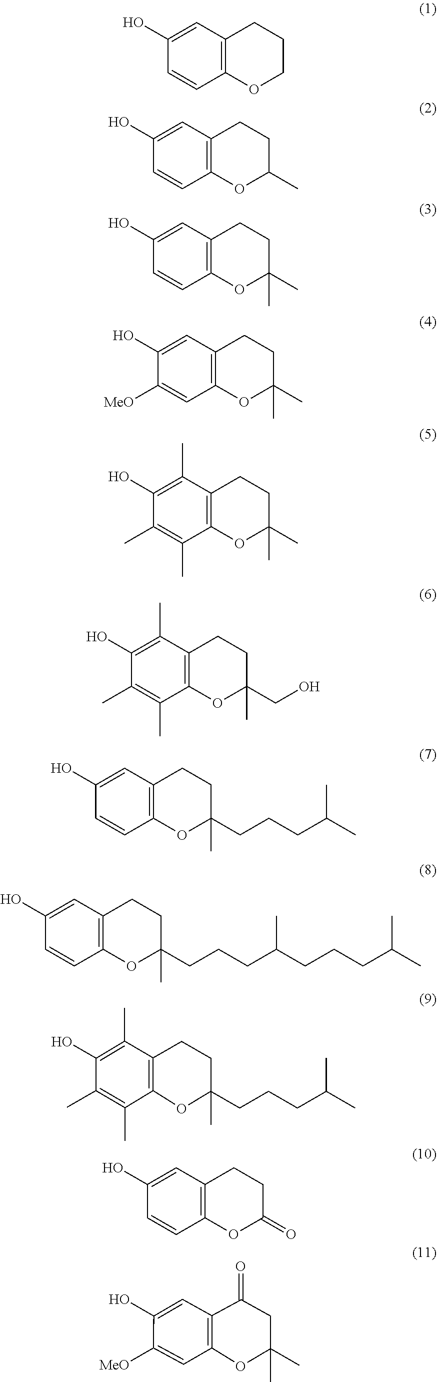

The compound of formula (8) (chemical name: 2-(4,8-dimethylnonyl)-2-methyl-chroman-6-ol) is a novel compound. Thus, this compound is also an object of the present invention.

The compounds of the present invention are efficient as antioxidants, preferably in feed and feed ingredients.

Non-limiting examples of feed are pet food, feed for aquatic animals, feed for terrestrial animals such as poultry and pigs, and feed for insects.

Non-limiting examples of feed ingredients are poultry meal, fish meal, insect meal and PUFA-containing oil.

“PUFA(s)” means polyunsaturated fatty acid(s) such as docosahexaenoic acid (“DHA”) and/or eicosapentaenoic acid (“EPA”) and/or docosapentaenoic acid (“DPA”) and/or oleic acid and/or stearidonic acid and/or linoleic acid and/or alpha-linolenic acid (“ALA”) and/or gamma-linolenic acid and/or arachidonic acid (“ARA”) and/or the esters of all of them, whereby the term “esters” encompasses monoglycerides, diglycerides and triglycerides as well as C₁₋₆-alkyl esters such as especially the methyl esters and the ethyl esters, whereby the triglycerides are often dominant.

DHA, EPA, ALA and stearidonic acid are omega-3 fatty acids, whereas linoleic acid, gamma-linolenic acid and ARA are omega-6 fatty acids.

The term “DPA” encompasses two isomers, the omega-3 fatty acid clupanodonic acid (7Z,10Z,13Z,16Z,19Z-docosapentaenoic acid) and the omega-6 fatty acid osbond acid (4Z,7Z,10Z,13Z,16Z-docosapentaenoic acid).

In accordance with the invention, the polyunsaturated fatty acid (PUFA) is preferably DHA and/or EPA and/or DPA and/or any ester thereof, more preferably the polyunsaturated fatty acid (PUFA) is preferably DHA and/or EPA and/or any ester thereof.

Examples of PUFA-containing oils are
marine oil, such as preferably fish oil,
microbial biomass containing polyunsaturated fatty acids and/or their esters (“microbial oil”), preferably containing high amounts of docosahexaenoic acid (“DHA”) and/or eicosapentaenoic acid (“EPA”) and/or docosapentaenoic acid (“DPA”) and/or their esters, and
oil containing high amounts of PUFAs and/or their esters, preferably containing high amounts of docosahexaenoic acid (“DHA”) and/or eicosapentaenoic acid (“EPA”) and/or docosapentaenoic acid (“DPA”) and/or their esters, extracted from microbial biomass, such as fungae (“fungal oil”) or algae (“algal oil”), and
plant oil with relatively high amounts of PUFAs and/or their esters, (“PUFA-containing plant oil”), such as e.g. canola seed oil, linseed/flaxseed oil, hempseed oil, pumpkin seed oil, evening primrose oil, borage seed oil, blackcurrent seed oil, sallow thorn/sea buckthorn oil, chia seed oil, argan oil and walnut oil.

Thus, in addition, the present invention is

(1) directed to the use of the compounds of formula (I) as antioxidants in feed, such as especially feed for aquatic animals, feed for terrestrial animals such as poultry, pigs and pets, and feed for insects; as well as
(2) directed to the use of the compounds of formula (I) as antioxidants in feed ingredients, such as especially poultry meal, fish meal, insect meal and PUFA-containing oil, and
(3) directed to feed, such as especially feed for aquatic animals, feed for terrestrial animals such as poultry, pigs and pets, and feed for insects, comprising such compounds of formula (I) and
(4) directed to feed ingredients, such as especially poultry meal, fish meal, insect meal and PUFA enriched oil, comprising such compounds of formula (I).

Thus, the present invention is directed to feed for aquatic animals comprising such compounds of formula (I) with the preferences as given above.

The present invention is also directed to feed for insects and terrestrial animals, e.g. pigs, poultry and pets, comprising such compounds of formula (I) with the preferences as given above.

Aquatic animals in the context of the present invention encompass farmed crustacea such as shrimp and carnivorous species of farmed fish such as salmons, rainbow trout, brown trout (Salmo trutta) and gilthead seabream.

Thus, the feed for aquatic animals comprising the compounds of formula (I) are especially fed to the aquatic animals as cited above.

I. Feed Ingredients

Feed ingredients are broadly classified into cereal grains, protein meals, fats and oils, minerals, feed additives, and miscellaneous raw materials, such as roots and tubers.

Further Antioxidants

The compounds of formula (I) can be used in combination with one or more other antioxidants as described below.

In an embodiment of the present invention the feed ingredients of the present invention additionally comprise a mixture of 2-tert-butyl-4-methoxyphenol and 3-tert-butyl-4-methoxyphenol, which is known under the name “BHA” (butylated hydroxyanisole).

In a further embodiment of the present invention the feed ingredients of the present invention additionally comprise ascorbyl palmitate.

In another embodiment of the present invention the feed ingredients of the present invention additionally comprise BHA and ascorbyl palmitate.

Instead of ascorbyl palmitate other esters of ascorbic acid such as the esters of ascorbic acid with linear C₁₂₋₂₀ alkanols, preferably the esters of ascorbic acid with linear C₁₄₋₁₈ alkanols, may also be used, so that further embodiments of the present invention are directed to feed ingredients that additionally comprise esters of ascorbic acid with linear C₁₂₋₂₀ alkanols, preferably esters of ascorbic acid with linear C₁₄₋₁₈ alkanols, more preferably ascorbyl palmitate, whereby optionally BHA may also be present.

The feed ingredients may also comprise additionally alpha-tocopherol and/or gamma-tocopherol, whereby either an ester of ascorbic acid with a linear C₁₂₋₂₀ alkanol with the preferences as given above or BHA or both may additionally be present.

The feed ingredients themselves are described in more detail below.

1. PUFA-Containing Oils

In the context of the present invention the term “PUFA-containing oil” encompasses
marine oil, such as especially fish oil,
microbial biomass containing polyunsaturated fatty acids (“PUFAs”), especially docosahexaenoic acid (“DHA”) and/or eicosapentaenoic acid (“EPA”) and/or docosapentaenoic acid (“DPA”) and/or their esters (“microbial oil”);
oil containing high amounts of PUFAs, especially containing high amounts of DHA and/or EPA and/or DPA and/or their esters extracted from microbial biomass as e.g., fungi (“fungal oil”) or algae (“algal oil”);
Plant oil with high amounts of PUFAs and/or their esters (“PUFA-containing plant oil”), such as e.g. canola seed oil, linseed/flaxseed oil, hempseed oil, pumpkin seed oil, evening primrose oil, borage seed oil, blackcurrent seed oil, sallow thorn/sea buckthorn oil, chia seed oil, argan oil and walnut oil.

The term “DHA” does not only encompass the acid but also derivatives thereof such as monoglycerides, diglycerides and triglycerides as well as C₁₋₆-alkyl esters such as the methyl and ethyl esters. The same applies for “EPA” and “DPA” and all the other PUFAs.

Fish oil and algal oil are common feed ingredients. Instead of fish oil and algal oil also the other PUFA-containing oils named above may be used as feed ingredients, i.e.:
microbial biomass containing PUFAs (“microbial oil”)
oil containing high amounts of PUFAs extracted from microbial biomass, such as especially fungal oil, and
plant oil with high amounts of PUFAs.

The above-mentioned feed ingredients may not only be used as alternative of fish oil and algal oil, but also in addition.

Examples of PUFA-containing oils that are used as feed ingredients are given below in more detail.

Marine Oil

Examples of suitable marine oils include, but are not limited to, Atlantic fish oil, Pacific fish oil, or Mediterranean fish oil, or any mixture or combination thereof.

In more specific examples, a suitable fish oil can be, but is not limited to, pollack oil, bonito oil, pilchard oil, tilapia oil, tuna oil, sea bass oil, halibut oil, spearfish oil, barracuda oil, cod oil, menhaden oil, sardine oil, anchovy oil, capelin oil, herring oil, mackerel oil, salmonid oil, tuna oil, and shark oil, including any mixture or combination thereof.

Other marine oils suitable for use herein include, but are not limited to, squid oil, cuttle fish oil, octopus oil, krill oil, seal oil, whale oil, and the like, including any mixture or combination thereof.

For stabilizing marine oil an amount of at least one compound of formula (I) ranging from 10 to 500 ppm, preferably ranging from 30 to 300 ppm, more preferably ranging from 100 to 250 ppm, based on the total amount of the marine oil, is usually sufficient. The same applies for the other PUFA-containing oils such as microbial oil, algal oil, fungal oil and PUFA-containing plant oil.

A commercially available example of marine oil is the fish oil “MEG-3” (Bleached 30S TG Fish oil) from DSM Nutritional Products, LLC (US) whose specification and composition is shown in Tables 1 and 2 below:

TABLE 1

| ANALYSIS | SPECIFICATIONS |
|---|---|
| Colour | Max. 6 Gardner Colour |
| Free Fatty Acid (as % Oleic) | Max. 0.4% |
| p-Anisidine Value | Max. 12 (at time of release) |
| Peroxide Value | Max. 3 milli equivalents/kg |
| | (at time of release) |
| % Moisture | Max. 0.05% |
| Cold Test | Remains clear at 0° C. for 3 hours |
| Cholesterol | Report Actual |
| TOTOX ((2 × Peroxide Value) + | Max. 20 |
| (p-Anisidine Value)) | |

The peroxide value is defined as the amount of peroxide oxygen per 1 kilogram of oil. Traditionally this is expressed in units of milliequivalents or meq/kg.

Winterization is part of the processing of fish oil, and it is performed to remove solid fat in the oil. The “cold test” is performed to check if any solid fat is present and precipitated in the oil when cooled to 0° C. within a specific period of time. In this fish oil (Product Code: FG30TG), any such precipitation is checked for 3 hours at 0° C.

TABLE 2

| Fatty Acid Profile | |
|---|---|
| EPA (A %) | Min. 18 |
| EPA mg/g (as TG) | Min. 170 |
| DHA (A %) | Min. 12 |
| DHA mg/g (as TG) | Min. 110 |
| EPA + DHA (A %) | Min. 30 |
| Total Omega 3 (A %) | Min. 34 |

“TG” = triglyceride;
“A %” = “area %” = area percentage by GC based on 24 peak analysis (meaning the 24 highest peaks have been analyzed)

Oil Containing High Amounts of PUFAs, Especially Containing High Amounts of DHA and/or EPA and/or DPA and/or their Esters, Extracted from Microbial Biomass as e.g., Fungi (“Fungal Oil”) or Algae (“Algal Oil”)

Algal Oil

“Algal oil” is an oil containing high amounts of DHA and/or EPA and/or DPA and/or their esters extracted from algae as microbial source/biomass.

An example of algal oil is the commercially available “Algal oil containing EPA+DPA” from DSM Nutritional Products, LLC (US) whose composition is shown in the Table 3 below:

TABLE 3

| Fatty Acid Profile | |
|---|---|
| DHA + EPA content, mg/g oil | 587 mg/g |
| DHA content, mg/g oil | 401 mg/g |
| EPA content, mg/g oil | 186 mg/g |
| TOTOX ((2 × Peroxide Value) + | 5 |
| (p-Anisidine Value)) | |
| Free Fatty Acid | 0.6% |
| Moisture | <0.05% |

A further example of a crude oil containing high amounts of DHA and/or EPA extracted from microbial sources as e.g., algae, is the oil extracted from Algae Schizochytrium Biomass, whose specification is given in the following Table 4.

TABLE 4

| Specification | Aqua (Base Product) |
|---|---|
| DHA + EPA, mg/g oil | minimal 500 mg/g |
| DHA content, mg/g oil | minimal 250 mg/g |
| | (at least 25% -> 40%) |
| EPA content, mg/g oil | minimal 100 mg/g |
| | (at least 10% -> 25%) |
| Minimal ratio EPA:DHA | 1:4 |
| Maximal ratio EPA:DHA | 1:1 |
| TOTOX ((2 × Peroxide Value) + | maximum 35 |
| (p-Anisidine Value)) | |
| Free fatty acid | maximal 5% |
| Moisture | maximal 0.75% |
| DPA n-3 (omega-3 | <6 |
| docosapentaenoic acid), % | |
| Arachidonic Acid, % | <2 |
| Stearic, % | <2.5 |
| Palmitic, % | <30 |
| Shelf life | 6 months at 25° C. |
| Total Fat | Record |
| Crude Fat | >92% |

Microbial Biomass Containing Polyunsaturated Fatty Acids (“PUFAs”), Especially Docosahexaenoic Acid and/or Eicosapentaenoic Acid and/or Docosapentaenoic Acid (“DPA”) and/or their Esters

The biomass preferably comprises cells which produce PUFAs hetero-trophically. According to the invention, the cells are preferably selected from algae, fungi, particularly yeasts, bacteria, or protists. The cells are more preferably microbial algae or fungi.

Suitable cells of oil-producing yeasts are, in particular, strains of Yarrowia, Candida, Rhodotorula, Rhodosporidium, Cryptococcus, Trichosporon and Lipomyces.

Oil produced by a microorganism or obtained from a microbial cell is referred to as “microbial oil”. Oil produced by algae and/or fungi is referred to as an algal and/or a fungal oil, respectively.

As used herein, a “microorganism” refers to organisms such as algae, bacteria, fungi, protist, yeast, and combinations thereof, e.g., unicellular organisms. A microorganism includes but is not limited to, golden algae (e.g., microorganisms of the kingdom Stramenopiles); green algae; diatoms; dinoflagellates (e.g., microorganisms of the order Dinophyceae including members of the genus Crypthecodinium such as, for example, Crypthecodinium cohnii or C. cohnii); microalgae of the order Thraustochytriales; yeast (Ascomycetes or Basidiomycetes); and fungi of the genera Mucor, Mortierella, including but not limited to Mortierella alpina and Mortierella sect. schmuckeri, and Pythium, including but not limited to Pythium insidiosum.

In one embodiment, the microorganisms of the kingdom Stramenopiles may in particular be selected from the following groups of microorganisms: Hamatores, Proteromonads, Opalines, Developayella, Diplophrys, Labrinthulids, Thraustochytrids, Biosecids, Oomycetes, Hypochytridiomycetes, Commotion, Reticulosphaera, Pelagomonas, Pelagococcus, Ollicola, Aureococcus, Parmales, Diatoms, Xanthophytes, Phaeophytes (brown algae), Eustigmatophytes, Raphidophytes, Synurids, Axodines (including Rhizochromulinales, Pedinellales, Dictyochales), Chrysomeridales, Sarcinochrysidales, Hydrurales, Hibberdiales, and Chromulinales.

In one embodiment, the microorganisms are from the genus Mortierella, genus Crypthecodinium, genus Thraustochytrium, and mixtures thereof. In a further embodiment, the microorganisms are from Crypthecodinium Cohnii. In a further embodiment, the microorganisms are from Mortierella alpina. In a still further embodiment, the microorganisms are from Schizochytrium sp. In yet an even further embodiment, the microorganisms are selected from Crypthecodinium Cohnii, Mortierella alpina, Schizochytrium sp., and mixtures thereof.

In a still further embodiment, the microorganisms include, but are not limited to, microorganisms belonging to the genus Mortierella, genus Conidiobolus, genus Pythium, genus Phytophthora, genus Penicillium, genus Cladosporium, genus Mucor, genus Fusarium, genus Aspergillus, genus Rhodotorula, genus Entomophthora, genus Echinosporangium, and genus Saprolegnia.

In an even further embodiment, the microorganisms are from microalgae of the order Thraustochytriales, which includes, but is not limited to, the genera Thraustochytrium (species include arudimentale, aureum, benthicola, globosum, kinnei, motivum, multirudimentale, pachydermum, proliferum, roseum, striatum); the genera Schizochytrium (species include aggregatum, limnaceum, mangrovei, minutum, octosporum); the genera Ulkenia (species include amoeboidea, kerguelensis, minuta, profunda, radiate, sailens, sarkariana, schizochytrops, visurgensis, yorkensis); the genera Aurantiacochytrium; the genera Oblongichytrium; the genera Sicyoidochytium; the genera Parientichytrium; the genera Botryochytrium; and combinations thereof. Species described within Ulkenia will be considered to be members of the genus Schizochytrium. In another embodiment, the microorganisms are from the order Thraustochytriales. In yet another embodiment, the microorganisms are from Thraustochytrium. In still a further embodiment, the microorganisms are from Schizochytrium sp.

In certain embodiments, the oil can comprise a marine oil. Examples of suitable marine oils are the ones as given above.

The biomass according to the invention preferably comprises cells, and preferably consists essentially of such cells, of the taxon Labyrinthulomycetes (Labyrinthulea, net slime fungi, slime nets), in particular, those from the family of Thraustochytriaceae. The family of the Thraustochytriaceae (Thraustochytrids) includes the genera Althomia, Aplanochytrium, Aurantiochytrium, Botryochytrium, Elnia, Japonochytrium, Oblongichytrium, Parietichytrium, Schizochytrium, Sicyoidochytrium, Thraustochytrium, and Ulkenia. The biomass particularly preferably comprises cells from the genera Aurantiochytrium, Oblongichytrium, Schizochytrium, or Thraustochytrium, more preferably from the genus Schizochytrium.

In accordance with the invention, the polyunsaturated fatty acid (PUFA) is preferably DHA and/or EPA and/or their esters as defined above.

The cells present in the biomass are preferably distinguished by the fact that they contain at least 20 weight-%, preferably at least 30 weight-%, in particular at least 35 weight-%, of PUFAs, in each case based on cell dry matter.

In a very preferred embodiment of the current invention, cells, in particular a Schizochytrium strain, is employed which produces a significant amount of EPA and DHA, simultaneously, wherein DHA is preferably produced in an amount of at least 20 weight-%, preferably in an amount of at least 30 weight-%, in particular in an amount of 30 to 50 weight-%, and EPA is produced in an amount of at least 5 weight-%, preferably in an amount of at least 10 weight-%, in particular in an amount of 10 to 20 weight-% (in relation to the total amount of lipid as contained in the cells, respectively).

Preferred species of microorganisms of the genus Schizochytrium, which produce EPA and DHA simultaneously in significant amounts, as mentioned before, are deposited under ATCC Accession No. PTA-10208, PTA-10209, PTA-10210, or PTA-10211, PTA-10212, PTA-10213, PTA-10214, PTA-10215.

DHA and EPA producing Schizochytrium strains can be obtained by consecutive mutagenesis followed by suitable selection of mutant strains which demonstrate superior EPA and DHA production and a specific EPA:DHA ratio. Any chemical or nonchemical (e.g. ultraviolet (UV) radiation) agent capable of inducing genetic change to the yeast cell can be used as the mutagen. These agents can be used alone or in combination with one another, and the chemical agents can be used neat or with a solvent.

Methods for producing the biomass, in particular, a biomass which comprises cells containing lipids, in particular PUFAs, particularly of the order Thraustochytriales, are described in detail in the prior art (see e.g. WO 91/07498, WO 94/08467, WO 97/37032, WO 97/36996, WO 01/54510). As a rule, the production takes place by cells being cultured in a fermenter in the presence of a carbon source and a nitrogen source, along with a number of additional substances like minerals that allow growth of the microorganisms and production of the PUFAs. In this context, biomass densities of more than 100 grams per litre and production rates of more than 0.5 gram of lipid per litre per hour may be attained. The process is preferably carried out in what is known as a fed-batch process, i.e. the carbon and nitrogen sources are fed in incrementally during the fermentation. When the desired biomass has been obtained, lipid production may be induced by various measures, for example by limiting the nitrogen source, the carbon source or the oxygen content or combinations of these.

In a preferred embodiment of the current invention, the cells are grown until they reach a biomass density of at least 80 or 100 g/l, more preferably at least 120 or 140 g/l, in particular at least 160 or 180 g/l (calculated as dry-matter content). Such processes are for example disclosed in U.S. Pat. No. 7,732,170.

Preferably, the cells are fermented in a medium with low salinity, in particular, so as to avoid corrosion. This can be achieved by using chlorine-free sodium salts as the sodium source instead of sodium chloride, such as, for example, sodium sulphate, sodium carbonate, sodium hydrogen carbonate or soda ash. Preferably, chloride is used in the fermentation in amounts of less than 3 g/l, in particular, less than 500 mg/l, especially preferably less than 100 mg/l.

PUFA-Containing Plant Oils: Plant Oils with Relatively High Amounts of PUFAs, Especially with High Amounts of DHA and/or EPA Such as e.g., Canola Seed Oil

The plant cells may, in particular, be selected from cells of the families Brassicaceae, Elaeagnaceae and Fabaceae. The cells of the family Brassicaceae may be selected from the genus Brassica, in particular, from oilseed rape, turnip rape and Indian mustard; the cells of the family Elaeagnaceae may be selected from the genus Elaeagnus, in particular, from the species Oleae europaea; the cells of the family Fabaceae may be selected from the genus Glycine, in particular, from the species Glycine max.

### EXAMPLES

Canola seed oil with a content of DHA of at least 9% by weight, of at least 12% by weight, of at least 15% by weight, or of at least 20% by weight, based on the total weight of the canola seed oil;
Canola seed oil with a content of EPA of at least 9% by weight, of at least 12% by weight, of at least 15% by weight, or of at least 20% by weight, based on the total weight of the canola seed oil.

Examples of PUFA-containing plant oils containing high amounts of other PUFAs than EPA and/or DHA and/or DPA and/or their esters are linseed/flaxseed oil, hempseed oil, pumpkin seed oil, evening primrose oil, borage seed oil, blackcurrent seed oil, sallow thorn/sea buckthorn oil, chia seed oil, argan oil and walnut oil.

2. Other Feed Ingredients

Poultry Meal/Chicken Meal

Poultry meal is a high-protein commodity used as a feed ingredient. It is made from grinding clean, rendered parts of poultry carcasses and can contain bones, offal, undeveloped eggs, and some feathers. Poultry meal quality and composition can change from one batch to another.

Chicken meal, like poultry meal, is made of “dry, ground, rendered clean parts of the chicken carcass” according to AAFCO and may contain the same ingredients as poultry meal. Chicken meal can vary in quality from batch to batch. Chicken meal costs less than chicken muscle meat and lacks the digestibility of chicken muscle meat.

Poultry meal contains preferably not less than 50 weight-% of crude protein, not less than 5 weight-% of crude fat, not more than 5 weight-% of crude fiber, not more than 40 weight-% of ash and not more than 15 weight-% of water, each based on the total weight of the poultry meal, whereby the total amount of all ingredients sums up to 100 weight-%.

More preferably poultry meal contains from 50 to 85 weight-% of crude protein, and from 5 to 20 weight-% of crude fat, and from 1 to 5 weight-% of crude fiber, and from 5 to 40 weight-% of ash, and from 5 to 15 weight-% of water, each based on the total weight of the poultry meal, whereby the total amount of all ingredients sums up to 100 weight-%.

For stabilizing poultry meal an amount of at least one compound of formula (I) ranging from 10 to 1000 ppm, preferably ranging from 30 to 700 ppm, more preferably ranging from 100 to 500 ppm, based on the total amount of the poultry meal, is usually sufficient.

The same amounts also apply for chicken meal.

Fish Meal

Fish meal contains preferably not less than 50 weight-% of crude protein, and not more than 20 weight-% of crude fat, and not more than 10 weight-% of crude fibers, and not more than 25 weight-% of ash, and not more than 15 weight-% of water, each based on the total weight of the fish meal, whereby the total amount of all ingredients sums up to 100 weight-%.

More preferably fish meal contains from 50 to 90 weight-% of crude protein and from 5 to 20 weight-% of crude fat, and from 1 to 10 weight-% of crude fibers, and from 5 to 25 weight-% of ash, and from 5 to 15 weight-% of water, each based on the total weight of the fish meal, whereby the total amount of all ingredients sums up to 100 weight-%.

For stabilizing fish meal an amount of at least one compound of formula (I) ranging from 10 to 2000 ppm, preferably ranging from 100 to 1500 ppm, more preferably ranging from 300 to 1000 ppm, based on the total amount of the fish meal, is usually sufficient.

Fish meal is a commercial product made from fish that is used primarily as a protein supplement in compound feed, especially for feeding farmed fish, crustacea, pigs and poultry, and companion animals such as cats and dogs.

A portion of the fish meal is made from the bones and offal left over from processing fish used for human consumption, while the larger percentage is manufactured from wild-caught, small marine fish. It is powder or cake obtained by drying the fish or fish trimmings, often after cooking, and then grinding it. If the fish used is a fatty fish it is first pressed to extract most of the fish oil.

The uses and need of fish meal are increasing due to the rising demand for fish, because fish has the best feed conversion rate of all farmed animals, can be produced well in developing countries and has a small size, i.e. can be slaughtered for preparing a meal, so that there is no need to store the fish. Furthermore, there are no religious constraints concerning the consumption of fish, fish is a source of high quality protein and it is easy to digest.

Fish meal is made by cooking, pressing, drying, and grinding of fish or fish waste to which no other matter has been added. It is a solid product from which most of the water is removed and some or all of the oil is removed. About four or five tons of fish are needed to manufacture one ton of dry fish meal.

Of the several ways of making fish meal from raw fish, the simplest is to let the fish dry out in the sun. This method is still used in some parts of the world where processing plants are not available, but the end-product is of poor quality in comparison with ones made by modern methods.

Currently, all industrial fish meal is usually made by the following process:

Cooking: A commercial cooker is a long, steam-jacketed cylinder through which the fish are moved by a screw conveyor. This is a critical stage in preparing the fishmeal, as incomplete cooking means the liquid from the fish cannot be pressed out satisfactorily and overcooking makes the material too soft for pressing. No drying occurs in the cooking stage.

Pressing: A perforated tube with increasing pressure is used for this process. This stage involves removing some of the oil and water from the material and the solid is known as press cake. The water content in pressing is reduced from 70% to about 50% and oil is reduced to 4%.

Drying: If the fish meal is under-dried, moulds or bacteria may grow. If it is over-dried, scorching may occur and this reduces the nutritional value of the meal.

The two main types of dryers are:

Direct: Very hot air at a temperature of about 500° C. is passed over the material as it is tumbled rapidly in a cylindrical drum. This is the quicker method, but heat damage is much more likely if the process is not carefully controlled.

Indirect: A cylinder containing steam-heated discs is used, which also tumbles the meal.

Grinding: This last step in processing involves the breakdown of any lumps or particles of bone.

The fish meal has to be transported long distances by ship or other vehicles to the various locations, where it is used.

Unmodified fish meal can spontaneously combust from heat generated by oxidation of the polyunsaturated fatty acids in the fish meal. Therefore, it has to be stabilized by antioxidants. Especially advantageous for this purpose are the compounds of formula (I) of the present invention.

Insect Meal

Insect meal has a high content of protein and is therefore, a valuable source of protein.

In general any insect may be manufactured to meal, but insects of special interest in the context of the present invention encompass black soldier flies (Hermetia species, commonly called BSF), mealworms (Tenebrio molitor), lesser mealworms (Alphitobius diaperinus), house cricket (Acheta domesticus, grasshoppers (Locusta migratoria), buffaloworms (Alphitobius diaperinus), cockroaches and domestic flies, whereby black soldier flies (Hermetia species, commonly called BSF), mealworms (Tenebrio molitor) and lesser mealworms (Alphitobius diaperinus) are more preferred.

For stabilizing insect meal an amount of at least one compound of formula (I) ranging from 10 to 1000 ppm, preferably ranging from 30 to 700 ppm, more preferably ranging from 100 to 500 ppm, based on the total amount of the insect meal, is usually sufficient.

### PREFERRED EMBODIMENTS OF THE PRESENT INVENTION

The compounds of formula (I) with the preferences as given above are not only suitable for stabilizing fish meal, but also for stabilizing feed ingredients and feed. Preferences for feed ingredients and feed are given above and also apply here.

Compounds of formulae (1), (2), (3), (4), (5), (6), (8) and (9) are especially preferred and amongst these compounds of formulae (1), (2), (3), (4), (6), (8) and (9) are even more preferred and compounds of formulae (1), (3), (4), (6), (8) and (9) are most preferred.

Compounds of formulae (3), (4), (5), and (6), preferably compounds of formulae (3) and (4), are especially suitable for stabilizing fish meal and thus prevent combustion of the fish meal and preserve its nutritional value.

The compounds especially suitable for stabilizing poultry meal are compounds of formulae (1), (2) and (3).

The compounds especially suitable for stabilizing pet food are compounds of formulae (1) and (3).

The compounds of formulae (4), (6), (8) and (9), preferably the compounds of formulae (4), (8) and (9), more preferably the compounds of formulae (4) and (8), are especially suitable for stabilizing marine oil, microbial oil and algal oil.

II. Feed

The compounds of formula (I) are not only suitable for stabilizing feed ingredients such as poultry meal, fish meal, insect meal and PUFA-containing oil, but also effective antioxidants for feed.

Feed (or ‘feedingstuff’) means any substance or product, including additives, whether processed, partially processed or unprocessed, intended to be used for oral feeding to animals.

Feed in the context of the present invention is feed for aquatic animals and for terrestrial animals, as well as feed for insects.

For stabilizing feed an amount of at least one compound of formula (I) ranging from 10 to 500 ppm, preferably ranging from 30 to 300 ppm, more preferably ranging from 100 to 250 ppm, based on the total amount of the feed, is usually sufficient.

Further Antioxidants

The compounds of formula (I) can be used in combination with one or more other antioxidants as described below.

In an embodiment of the present invention the feed of the present invention additionally comprises a mixture of 2-tert-butyl-4-methoxyphenol and 3-tert-butyl-4-methoxyphenol, which is known under the name “BHA” (butylated hydroxyanisole).

In a further embodiment of the present invention the feed of the present invention additionally comprises ascorbyl palmitate.

In another embodiment of the present invention the feed of the present invention additionally comprises BHA and ascorbyl palmitate.

Instead of ascorbyl palmitate other esters of ascorbic acid such as the esters of ascorbic acid with linear C₁₂₋₂₀ alkanols, preferably the esters of ascorbic acid with linear C₁₄₋₁₈ alkanols, may also be used, so that further embodiments of the present invention are directed to feed that additionally comprises esters of ascorbic acid with linear C₁₂₋₂₀ alkanols, preferably esters of ascorbic acid with linear C₁₄₋₁₈ alkanols, more preferably ascorbyl palmitate, whereby optionally BHA may also be present.

The feed may also comprise additionally alpha-tocopherol and/or gamma-tocopherol, whereby either an ester of ascorbic acid with a linear C₁₂₋₂₀ alkanol with the preferences as given above or BHA or both may additionally be present.

The feed itself is described in more detail below.

Feed for Poultry

The feed for poultry differs from region to region. In the following Tables 5 and 6 typical examples for diets in Europe and Latin America are given. These diets include cereals such as wheat, rye, maize/corn, minerals such as NaCl, vegetable oils such as soya oil, amino acids and proteins.

TABLE 5
European diet

| | Starter Period | Grower Period |
|---|---|---|
| Ingredients (%) | (day 0-21) | (day 22-36) |
| Wheat | 20.00 | 22.50 |
| Rye | 12.00 | 12.00 |
| Soybean meal | 34.00 | 28.50 |
| Maize | 27.00 | 28.50 |
| Vegetable Oil | 3.10 | 4.20 |
| NaCl | 0.10 | 0.10 |
| DL Methionine | 0.24 | 0.24 |
| L-Lysine | 0.15 | 0.15 |
| Limestone | 0.85 | 0.85 |
| Dicalcium Phosphate | 1.50 | 1.90 |
| Vitamin & Mineral mix | 1.00 | 1.00 |
| Coccidiostat (Avatec) | 0.06 | 0.06 |
| TiO₂ | — | 0.10 |
| calculated Provision | | |
| apparent metabolizable | 12.5 | 12.90 |
| energy, MJ/kg | | |
| apparent metabolizable | 2986 | 3082 |
| energy, kcal/kg | | |
| crude Protein, % | 21.2 | 19.1 |
| Methionine + Cysteine, % | 0.89 | 0.83 |
| Lysine, % | 1.23 | 1.09 |
| Calcium, % | 0.83 | 0.91 |
| total phosphorus, % | 0.68 | 0.73 |
| available phosphorus, % | 0.35 | 0.40 |

TABLE 6
Latin American diet

| Ingredients (%) | Starter | Grower |
|---|---|---|
| Corn | 53.0 | 57.1 |
| Soybean meal | 38.5 | 34.2 |
| Calcium | 0.70 | 0.70 |
| Phosphorus | 2.40 | 2.00 |
| NaHCO₃ | 0.23 | 0.24 |
| NaCl | 0.20 | 0.20 |
| Methionine | 0.30 | 0.10 |
| Lysine | 0.21 | 0.00 |
| Soya Oil | 3.50 | 4.50 |
| Premix | 1.00 | 1.00 |
| Calculated provision (%) | | |
| Crude protein | 22.4 | 20.4 |
| apparent metabolizable energy, (MJ/kg) | 12.7 | 13.2 |
| apparent metabolizable energy, (kcal/kg) | 3034 | 3154 |
| Total phosphorus | 0.86 | 0.76 |
| Calcium | 1.00 | 0.85 |
| Available phosphorus | 0.44 | 0.38 |
| d-Lysine | 1.25 | 0.98 |
| d-Methionine + Cysteine | 0.91 | 0.68 |
| d-Threonine | 0.77 | 0.71 |
| Na | 0.18 | 0.18 |
| Cl | 0.20 | 0.19 |

Pet Food

Pet foods are formulated to meet nutrient specifications using combinations of multiple ingredients to meet the targeted nutrient specification.

Poultry meal e.g. is an ingredient that is commonly found in Dog and Cat foods.

The nutrient specifications for a complete and balanced dog or cat food will meet or exceed the guidelines provided by AAFCO (American Association of Feed Control Officials). The ingredient composition of pet-food can include any legal feed ingredient so number of combinations are not quite infinite but close. Some examples of ingredient used in dog and cat foods can be found in Table 7 below:

TABLE 7

| Ingredient Class/Ingredient | Use rates |
|---|---|
| 1 ANIMAL MEALS | 10-35% |
| Chicken | |
| Turkey | |
| Duck | |
| Poultry Br-Product | |
| Lamb | |
| Venison | |
| Beef | |
| Pork | |
| Meat & Bone | |
| Fish | |
| 2 FRESH MEATS | 3-20% |
| Chicken | |
| Turkey | |
| Duck | |
| Lamb | |
| Venison | |
| Beef | |
| Pork | |
| Fish | |
| 3 VEGETABLE PROTEINS | 8-20% |
| Soybean Meal | |
| Corn Gluten Meal | |
| Pea Protein | |
| Potato Protein | |
| Soy Protein Conc/Isolates | |
| 4 GRAINS | 0-70% |
| Corn/Maize | |
| Wheat | |
| Brown Rice/Brewers Rice | |
| Oatmeal/Oat Groats | |
| Barley | |
| Millet | |
| Milo/Sorghum | |
| Rye | |
| Corn Gluten Feed | |
| Wheat Middlings | |
| 5 FIBER SOURCES | 2-8% |
| Beet Pulp | |
| Corn Bran | |
| Wheat Bran | |
| Cellulose | |
| Tomato Ponace | |
| Potato Fiber | |
| Pea Fiber | |
| 6 FATS & OILS | 1-15% |
| Animal Fat | |
| Poultry Fat | |
| Chicken Fat | |
| Beef Tallow | |
| Sunflower Oil | |
| Canola Oil | |
| 7 MICRONUTRIENTS | 0.10-1% |
| Vitamins | |
| Minerals | |
| Others (e.g. Fructooligosaccharides | |
| (FOS) used as a pre-biotic) | |
| 8 PALATANTS (FLAVORS) | 0-5% |
| 9 Other non-basic ingredients | |
| Dried Egg Product | 1-15% |
| Fish Oil | 0.5-2% |
| Fish Meal | 1-4% |
| Flaxseed | 1-4% |
| Dried Peas | 5-30% |
| Dried Chickpeas | 5-30% |
| Dried Lentils | 5-10% |
| Dried Potatoes | 5-20% |
| Dried Sweet Potatoes | 5-20% |
| Tapioca Starch | 5-15% |
| Potato Starch | 5-15% |
| Pea Starch | 5-15% |

For stabilizing pet food an amount of at least one compound of formula (I) ranging from 10 to 500 ppm, preferably ranging from 30 to 300 ppm, more preferably ranging from 100 to 250 ppm, based on the total amount of the pet food, is usually sufficient.

Feed for Fish

A typical example of feed for fish comprises the following ingredients, whereby all amounts are given in weight-%, based on the total weight of the feed for fish:
Fish meal in an amount ranging from 5 to 15 weight-%, preferably fish meal in said amount comprising the compounds of formula (I) of the present invention;
fish hydrolysates in an amount ranging from 0 to 5 weight-%;
vegetable proteins in an amount ranging from 30 to 45 weight-%;
binders, mainly starch, in an amount ranging from 9 to 12 weight-%;
micro-ingredients such as vitamins, choline, minerals, mono calcium phosphate (“MCP”) and/or amino acids in an amount ranging from 3 to 6 weight-%;
marine oil in an amount ranging from 5 to 10 weight-%, preferably marine oil in said amount comprising the compounds of formula (I) of the present invention;
vegetable oil in an amount ranging from 20 to 25 weight-%, preferably vegetable oil in said amount comprising the compounds of formula (I) of the present invention;
and whereby the amount of all ingredients sum up to 100 weight-%.

For stabilizing feed for fish an amount of at least one compound of formula (I) ranging from 10 to 1000 ppm, preferably ranging from 30 to 700 ppm, more preferably ranging from 100 to 500 ppm, based on the total amount of the feed for fish, is usually sufficient.

The invention is now further illustrated in the following non-limiting examples.

### EXAMPLES

### Examples 1-10: Syntheses of Compounds of Formulae (1) to (4) and (6) to (11)

Compound of formula (5) (PMC=Pentamethylchromanol, CAS 950-99-2) is commercial and is e.g. purchased from Aldrich (catalog #430676).

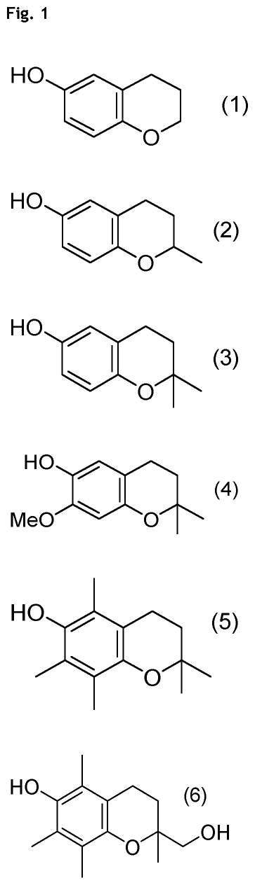

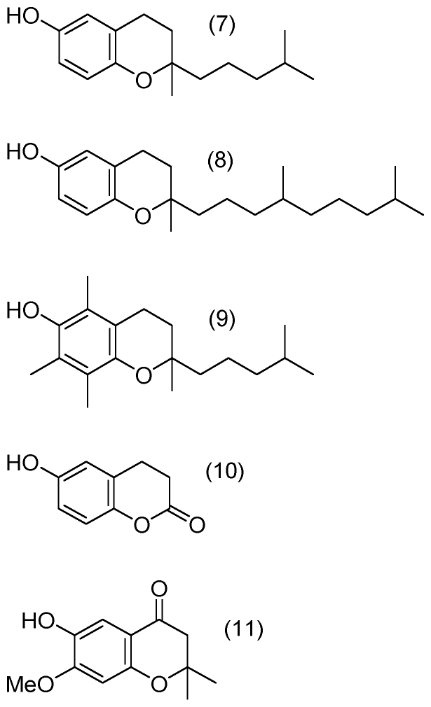

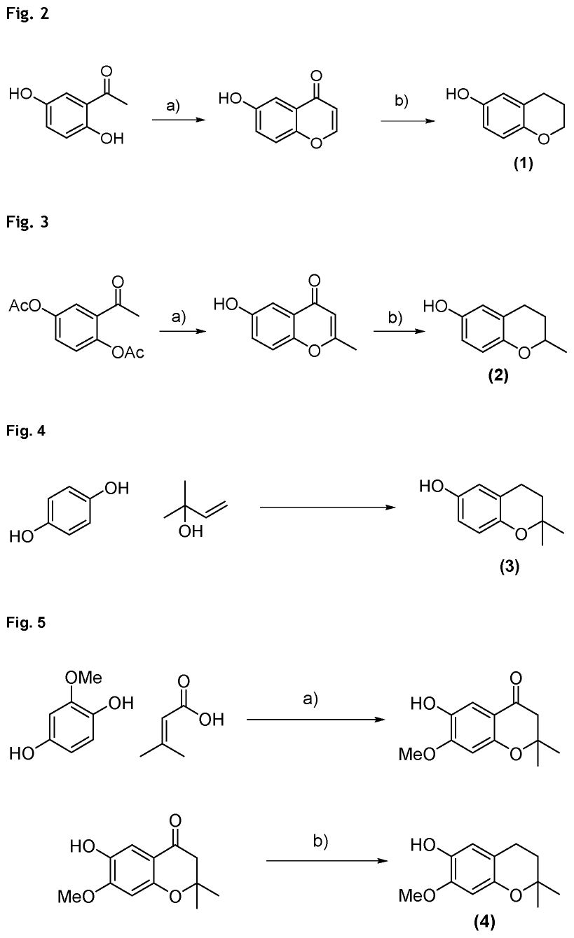

### Example 1: Synthesis of Compound of Formula (1) (6-Chromanol) (See FIG. 2)

First step a): HC(OEt)₃, 70% HClO₄-1) room temperature, 1 hour; 2) 100° C., 5 minutes in water, then room temperature (yield: 95%);

Second step b): H₂, Pd/C, 40° C., 8 hours, 5 bar (yield: 56%).

The procedure is described by J. C. Jaen, L. D. Wise, T. G. Heffner, T. A. Pugsley, L. T. Meltzer: “Dopamine autoreceptor agonists as potential antipsychotics. 2. (Aminoalkoxy)-4H-1-benzopyran-4-ones.” in J. Med. Chem. 1991, 34, 248-256. It is followed for the first step a), which furnishes the intermediate in high yield and good selectivity. The crude intermediate enone is then hydrogenated using Pd/C in a second step b), furnishing chroman-6-ol in 53% yield over two steps.

### Example 2: Synthesis of Compound of Formula (2) (2-Methyl-6-Chromanol) (see FIG. 3)

First step a): 1) KOtBu, tetrahydrofuran; 2) CuCl₂; 3) NaOH; 24 hours at room temperature; yield: 46%.

Second step b): H₂, Pd/C; EtOH; 70° C., 7 days, 10 bar, yield: 20%.

Starting from 2-acetyl-1,4-phenylene diacetate a base-mediated intramolecular aldol condensation furnishes the enone intermediate as described before (A. O. Termath, Stereoselektive Totalsynthese von a-Tocopherol durch Cu-katalysierte asymmetrische 1,4-Addition an ein Chromenon, Dissertation, Universität zu Köln, ISBN 978-3-8439-1407-9, Verlag Dr. Hut, München, 2013; Chapter 6.2.2, page 196-197.). The subsequent hydrogenation, using similar conditions as for compound of formula (1) above, furnishes the product in 20% yield.

### Example 3: Synthesis of Compound of Formula (3) (2,2-Dimethyl-6-Chromanol) (See FIG. 4)

Conditions: Formic acid (80%), 110° C., 4 h; yield 37%.

Compound of formula (3) is prepared following the literature procedure: Q.

Wang, X. She, X. Ren, J. Ma, X. Pan. “The First Asymmetric Total Synthesis of Several 3,4-Dihydroxy-2,2-Dimethyl-Chroman Derivatives.”, Tetrahedron: Asymmetry 2004, 15, 29-34.

### Example 4: Synthesis of Compound of Formula (4) (7-Methoxy-2,2-Dimethyl-6-Chromanol) (See FIG. 5)

Compound of formula (4) (=Lipochroman-6®, CAS 83923-51-7) is prepared according to literature procedures (see FIG. 5 for the reaction scheme).

First Step a): MeSO₃H (solvent), P₂O₅ (50 mol-%, based on 2-methoxy-1,4-hydroquinone), yield: 91%. (F. Camps, J. Coll, A. Messeguer, M. A. Pericás, S. Ricart, W. S. Bowers, D. M. Soderlund, Synthesis 1980, 725-727: “An Improved Procedure for the Preparation of 2,2-Dimethyl-4-chromanones.”)

Second Step b): LiAlH₄, Et₂O, yield: 87%.

(P. Anastasis, P. E. Brown, J. Chem. Soc., Perkin Trans. 1 1982, 2013-2018: “Analogues of antijuvenile hormones”.)

### Example 5: Synthesis of Compound of Formula (6) (2-Hydroxymethyl-2,5,7,8-Tetramethyl-6-Chromanol)

Compound of formula (6) (Trolol, CAS 79907-49-6) is prepared according to the following literature procedure: J.-W. Huang, C.-W. Shiau, J. Yang, D.-S. Wang, H.-C. Chiu, C.-Y. Chen, C.-S. Chen. Development of Small-Molecule Cyclin D1-Ablative Agents. J. Med. Chem. 2006, 49, 4684-4689.

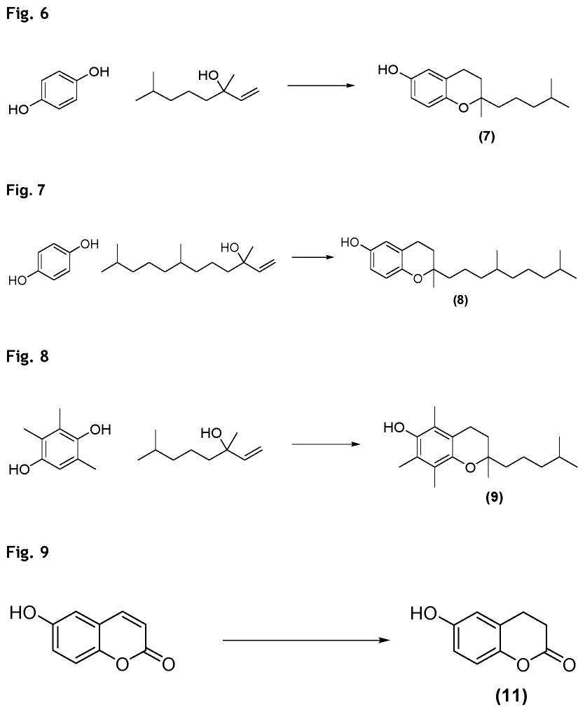

### Example 6: Synthesis of Compound of Formula (7) (2-(4-Methylpentyl)-2-Methyl-Chroman-6-Ol) (See FIG. 6)

A 1500 mL 4-necked flask with magnetic stirrer, oil bath, thermometer and argon supply was charged with hydroquinone (95.0 g, 864 mmol, 4.0 mol equiv.), 7-dimethyloct-1-en-3-ol (34.0 g, 216 mmol, 1.0 mol equiv.) and dissolved in ethylene carbonate (EC, 400 mL) and heptane (300 mL), forming a 2-phase system. Then, p-toluenesulfonic acid monohydrate (0.38 g, 2.16 mmol, 1 mol %) was added and the mixture was heated to reflux. After 1 h, deionized water (500 mL) was added to the reaction mixture and the hot reaction phases were separated. The lower EC phase was extracted twice with a total of heptane (600 mL). The combined heptane phases were dried over Na₂SO₄ and concentrated in vacuo (40° C./50-20 mbar). The residue was purified by column chromatography (eluent: heptane/EtOAc 95:5 to 85:15, w/w). The combined pure fractions were concentrated in vacuo (40° C./200-10 mbar) and dried under high vacuum at 45° C., furnishing 2-(4-methylpentyl)-2-methyl-chroman-6-ol as colorless oil (31.7 g, 97% purity by qNMR, 124 mmol, 58% yield).

HRMS: Calcd. for C₁₆H₂₄O₂ (M⁺) 248.1776, found 248.1800.

¹H NMR (300 MHz, CHLOROFORM-d) δ 0.88 (d, J=6.6 Hz, 6H), 1.12-1.23 (m, 2H), 1.27 (s, 3H), 1.33-1.46 (m, 2H), 1.46-1.62 (m, 3H), 1.64-1.89 (m, 2H), 2.71 (t, J=6.9 Hz, 2H), 4.51 (s, 1H, OH), 6.54-6.63 (m, 2H), 6.66 (d, J=8.9 Hz, 1H) ppm.

¹³C NMR (75 MHz, CHLOROFORM-d) δ 21.4 (1 C), 22.4 (1 C), 22.6 (2 C), 24.1 (1 C), 27.9 (1 C), 30.9 (1 C), 39.4 (1 C), 39.7 (1 C), 75.9 (1 C), 114.4 (1 C), 115.4 (1 C), 117.8 (1 C), 122.0 (1 C), 147.9 (1 C), 148.4 (1 C) ppm.

### Example 7: Synthesis of Compound of Formula (8) (2-(4,8-Dimethylnonyl)-2-Methyl-Chroman-6-Ol) (See FIG. 7)

A 200 mL 4-necked flask equipped with magnetic stirrer, oil bath, thermometer and argon supply was charged with 1,4-hydroquinone (12 g, 109 mmol, 4.0 mol equiv.), 3,7,11-trimethyldodec-1-en-3-ol (6.39 g, 27.2 mmol, 1.0 mol equiv.) and dissolved in ethylene carbonate (EC, 50 mL) and heptane (50 mL) forming a 2-phase system. Then, p-toluenesulfonic acid monohydrate (0.10 g 0.54 mmol, 2 mol %) was added and the mixture heated to reflux. After 90 min, the reaction mixture was cooled to 80° C. and the phases were separated. The lower EC phase was extracted with heptane (25 mL). The combined organic phases were dried over sodium sulfate and concentrated in vacuo (40° C./50-20 mbar). The residue was purified by column chromatography (eluent heptane/EtOAc 95:5 to 85:15 w/w). The combined pure fractions were concentrated in vacuo (40° C./200-10 mbar) and dried under high vacuum at 40° C., furnishing 2-(4,8-dimethylnonyl)-2-methyl-chroman-6-ol as light beige oil (4.95 g, 98.4% purity by qNMR, 15.3 mmol, 56% yield).

HRMS: Calcd. for C₂₁H₃₄O₂ (M⁺) 318.2559, found 318.2370.

¹H NMR (300 MHz, CHLOROFORM-d) δ 0.86 (d, J=˜6 Hz, 3H), superimposed by 0.88 (d, J=6.6 Hz, 6H), 1.04-1.46 (m, 11H), superimposed by 1.27 (s, 3H), 1.46-1.67 (m, 3H), 1.70-1.87 (m, 2H), 2.71 (t, J=6.8 Hz, 2H), 4.54 (br s, 1H, OH), 6.53-6.62 (m, 2H), 6.66 (d, J=8.5 Hz, 1H) ppm.

¹³C NMR (75 MHz, CHLOROFORM-d) δ 19.6, 21.1, 22.3, 22.6, 22.7, 24.1, 24.8, 28.0, 30.8, 30.9, 32.7, 37.3, 37.5, 39.3, 39.78, 39.82, 76.0, 114.4, 115.4, 117.8, 122.0, 147.9, 148.4 ppm.

### Example 8: Synthesis of Compound of Formula (9) (2,5,7,8-Tetramethyl)-2-(4-Methylpentyl)-Chroman-6-Ol) (See FIG. 8)

A 1.5 L 4-necked flask equipped with mechanical stirrer, oil bath, thermometer and argon supply was charged with 2,3,5-trimethyl-1,4-hydroquinone (134 g, 853 mmol, 4.0 mol equiv.), 3,7,11-trimethyldodec-1-en-3-ol (34 g, 213 mmol, 1.0 mol equiv.) and dissolved in ethylene carbonate (EC, 300 mL) and heptane (300 mL) forming a 2-phase system. Then, p-toluenesulfonic acid monohydrate (0.37 g 0.54 mmol, 1 mol %) was added and the mixture heated to reflux. After 90 min, the reaction mixture was cooled to 60° C. and the phases were separated. The lower EC phase was extracted with heptane (2×250 mL). The combined heptane phases were extracted with water (500 mL), dried over magnesium sulfate and concentrated in vacuo (40° C./50-20 mbar). The residue was purified by column chromatography (eluent heptane/EtOAc 95:5 to 80:20 w/w). The combined pure fractions were concentrated in vacuo (40° C./200-10 mbar) and dried under high vacuum at 40° C., furnishing 2,5,7,8-tetramethyl)-2-(4-methylpentyl)-chroman-6-ol as off-white solid (26.8 g, 98.9% purity by qNMR, 91 mmol, 43% yield).

Spectral data are in agreement with literature data: L. Rotolo, E. C. Gaudino, D. Carnaroglio, A. Barge, S. Tagliapietra, G. Cravotto, RSC Adv. 2016, 6, 63515-63518.

### Example 9: Synthesis of Compound of Formula (11) (6-Hydroxychroman-2-One) (See FIG. 9)

A 100 mL 3-necked round-bottom flask equipped with magnetic stirrer and septa was charged with 6-hydroxycoumarin (1.38 g, 8.17 mmol), THF (30 mL). Pd/C (10% Pd on C, 208 mg, 0.20 mmol, 2.4 mol %) was then added to this suspension. The flask was then inertized with argon (3× evacuation Et flush with argon) and finally a hydrogen balloon was attached. The reaction was stirred at room temperature for 18 h and monitored by HPLC (full conversion after 18 h). The catalyst was filtered off and washed with THF (3×5 mL). The filtrate was concentrated in vacuo (60° C./2 mbar), furnishing 1.41 g of crude product. Thus, the residue was suspended in Et₂O (6 g) and stirred for 18 h at room temperature. The solid was isolated by filtration, washed with Et₂O (2×3 mL) and dried at 60° C./20 mbar for 5 h. The product was obtained as colorless solid (1.25 g, 97.1% by qNMR, 91% yield).

mp 161-162° C.

¹H NMR (300 MHz, DMSO-d₆) δ 2.66-2.75 (m, 2H), 2.82-2.94 (m, 2H), 6.61-6.67 (m, 2H), 6.82-6.89 (m, 1H), 9.33 (s, 1H) ppm.

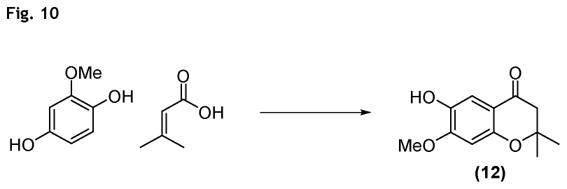

### Example 10: Synthesis of Compound of Formula (12) (6-Hydroxy-7-Methoxy-2,2-Dimethyl-Chroman-4-One) (See FIG. 10)

Compound of formula (12) is an intermediate in the synthesis of compound of formula (4) as described in example 4.

Conditions: MeSO₃H (solvent), P₂O₅ (50 mol-%, based on 2-methoxy-1,4-hydroquinone), yield: 91%. (F. Camps, J. Coll, A. Messeguer, M. A. Pericás, S. Ricart, W. S. Bowers, D. M. Soderlund, Synthesis 1980, 725-727: “An Improved Procedure for the Preparation of 2,2-Dimethyl-4-chromanones.”)

### Example 11: Antioxidant Activities in Pet Food, Poultry Meal and Fish Meal

Compounds of formulae (1) to (6) were tested in pet food, poultry meal and/or fish meal and their corresponding antioxidant efficacy values (“EV”) were determined subsequently.

Determination of the Antioxidant Efficacy Value “EV”

Oxidative stability was assessed using an Oxipres (Mikrolab Aarhus A/S, Hojbjerg, Denmark). The ML OXIPRES® is designed to monitor the oxidation of heterogeneous products. Consumption of oxygen results in a pressure drop which is measured by means of pressure transducers. The samples are heated to accelerate the process and shorten the analysis time (Mikrolab Aarhus 2012).

Sample weights were 50 g. They were loaded into the Oxipres vessels and placed inside the stainless-steel pressure vessel and sealed. The pressure vessels were purged with pure oxygen and filled to an initial oxygen pressure of 5 bar and maintained at 70° C. during measurement (D. Ying, L. Edin, L. Cheng, L. Sanguansri, M. A. Augustin, LWT—Food Science and Technology 2015, 62, 1105-1111: “Enhanced oxidative stability of extruded product containing polyunsaturated oils.”).

The oxygen pressure was recorded as function of time. After sample load and temperature rise the pressure in the device is increasing within 10 hours up to the starting pressure. Thereafter it is decreasing. Consequently, the starting pressure is considered as being the pressure after 10 hours. The analysis ends after 130 hours at 70° C. The oxygen consumption ‘O₂’ of the tested sample is calculated as follows:

$$O 2 consumption ( as \% ) = 1 - [ \frac{Pressure after 130 hours in Oxipres}{Pressure after 10 hours in Oxipres} ]$$

TABLE 8
Oxipres performance of the three matrices

| | Matrix 1 | Matrix 2 | Matrix 3 |
|---|---|---|---|
| | Pet food | Poultry meal | Fish meal |
| Oxipres results—O₂ | 25% | 32% | 31% |
| consumption | | | |
| CV (=coefficient of | 19% | 13% | 9% |
| variation) | | | |

A factor of protection called ‘EV’ (Efficacy Value) was developed to quantify with a relative number (relative to BHT) the antioxidant effect of the tested candidates. EV was calculated as follows:

$${EV}_{AOX candidate} = \frac{1}{( O 2 {consumption}_{AOX candidates} \text{/} O 2 {consumption}_{BHT} )}$$

EV, being relative to BHT (3,5-di-tert-butyl-4-hydroxy-toluene) (EV=1), makes it possible to compare the antioxidant compounds in a defined feed application. Here pet food, poultry meal and fish meal have been used as feed application with the composition as given in the following table 9.

TABLE 9

| | | | Poultry | Fish |
|---|---|---|---|---|
| | | Pet food | meal | meal |
| Parameters analyzed | Amount | Matrix 1 | Matrix 2 | Matrix 3 |
| Crude Protein | g/100 g | 24 | 56 | 68 |
| Total fat | g/100 g | 9.8 | 19.4 | 12.4 |
| Saturated fatty acids | g/100 g | 32.8 | 30.8 | 20.5 |
| | Fat | | | |
| Mono unsaturated | g/100 g | 38.1 | 43.5 | 31.0 |
| fatty acids | Fat | | | |
| Poly unsaturated | g/100 g | 19.6 | 17.9 | 28.3 |
| fatty acids | Fat | | | |
| Omega 3 | g/100 g | 3.76 | 0.72 | 25.9 |
| | Fat | | | |
| Omega 6 | g/100 g | 15.8 | 17.1 | 2.36 |
| | Fat | | | |
| Saturated fatty acids | g/100 g | 3.21 | 5.97 | 2.55 |
| Mono unsaturated fatty acids | g/100 g | 3.73 | 8.42 | 3.85 |
| Poly unsaturated fatty acids | g/100 g | 1.91 | 3.45 | 3.50 |
| Omega 3 | g/100 g | 0.367 | 0.139 | 3.22 |
| Omega 6 | g/100 g | 1.55 | 3.32 | 0.294 |
| Omega 3 + 6 | g/100 g | 1.92 | 3.46 | 3.51 |
| Unsaturated Fatty acids | g/100 g | 7.56 | 15.33 | 10.86 |
| Moisture content | % | 8.0 | 4.8 | 7.3 |
| water activity | | 0.49 | 0.42 | 0.53 |
| pH | | 7.8 | 7.6 | 7.4 |

Each of the compounds of formulae (1) to (6) were mixed into each matrix 1, 2 or 3 (pet food, poultry meal, fish meal) in an equimolar ratio compared to BHT. Batches of 200 g feed were produced in order to handle a minimum of 30 mg of antioxidant. First, a 1% pre-dilution of the antioxidant with the feed material was made. Then this pre-dilution was added to the final batch, mixed, sieved (1.25 mm sieve) and mixed using a turbula mixer. Thereafter 55 g of the final batch were packed into polyethylene bags, and stored at 4° C. until start of the Oxipres assay. Spare sample were stored at 4° C. Compound of formula (3) was measured in all three matrices. Compound of formula (1) was measured in matrix 1 and 2. Compound of formula (2) was measured in matrix 2. Compounds of formulae (4), (5) and (6) were measured in matrix 3. An efficacy value 0.7 has been considered as acceptable, an efficacy value ≥ the efficacy value of alpha-tocopherol as good, and an efficacy value ≥ the efficacy value of BHT as very good.

The results in pet food are shown in the following table 10. Compound of formula (1) and compound of formula (3) showed a higher efficacy value than alpha-tocopherol (EV=0.77).

TABLE 10

| | Compound of formula (1) | Compound of formula (3) |
|---|---|---|
| | chroman-6-ol 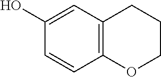 [SMILES: Oc1ccc2c(c1)CCCO2] | 2,2-dimethyl-chroman-6-ol 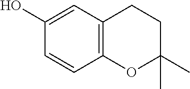 |
| EV in | 0.98 | 0.95 |
| pet food | | |

The results in poultry meal are shown in the following table 11. Compounds of formulae (1) to (3) showed a higher efficacy value than alpha-tocopherol (EV=0.74) and a higher efficacy value than BHT (EV=1) in poultry meal.

TABLE 11

| | Compound of | Compound of | Compound of |
|---|---|---|---|
| | formula (1) | formula (2) | formula (3) |
| | chroman-6-ol 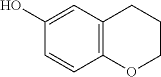 [SMILES: Oc1ccc2c(c1)CCCO2] | 2-methyl-chroman-6-ol 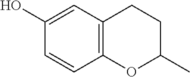 | 2,2-dimethyl- chroman-6-ol 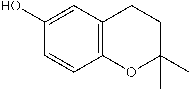 |
| EV in | 1.12 | 1.09 | 1.17 |
| poultry | | | |
| meal | | | |

The results in fish meal are shown in the following table 12. All 4 compounds showed a higher efficacy value than alpha-tocopherol (EV=0.88). The compounds of formulae (4), (5) and (6) even showed a higher efficacy value than BHT (EV=1) in fish meal.

TABLE 12

| | Compound | | | |
|---|---|---|---|---|
| | of formula | Compound of | Compound of | Compound of |
| | (3) | formula (4) | formula (5) | formula (6) |
| | 2,2-dimethyl- chroman-6-ol 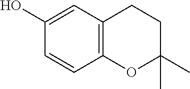 | 7-methoxy-2,2- dimethylchroman-6-ol 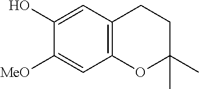 | 2,2,5,7,8- pentamethyl-chroman-6-ol 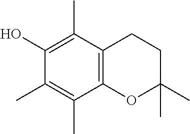 [SMILES: Cc1c(C)c2c(c(C)c1O)CCC(C)(C)O2] | 2-hydroxymethyl- 2,5,7,8-tetramethyl- chroman-6-ol 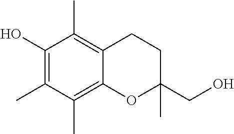 [SMILES: Cc1c(C)c2c(c(C)c1O)CCC(C)(CO)O2] |
| EV in | 0.96 | 1.05 | 1.08 | 1.03 |
| Fish | | | | |
| meal | | | | |

### Example 12: Antioxidant Activities of Compounds of Formulae (4), (6), (8) and (9) in Fish Oil

The compounds of formulae (4), (6), (8) and (9) have been tested. The blank oil, i.e. oil without any antioxidant, and oil containing “MNT” have been used as benchmark. Any compound better in antioxidant activity than the blank oil indicates that it has antioxidant activity. The comparison with MNT gives an indication about the amount of the antioxidant effect, relative to the activity of MNT.

“MNT” are mixed natural tocopherols commercially available as e.g., “Tocomix 70 IP” from AOM (Buenos Aires, Argentina). Tocomix 70 IP comprises d-alpha-tocopherol, d-beta-tocopherol, d-gamma-tocopherol and d-delta-tocopherol, whereby the total amount of tocopherols is at least 70.0 weight-% and the amount of non-alpha tocopherols is at least 56.0 weight-%.

The compounds of formulae (4), (6), (8) and (9) were evaluated primarily for their oxidative stability by the Oil Stability Index (OSI) measurements. Two different levels of these antioxidants (0.5 and 2 mg/g) were used in 5 g of natural fish oil (Product code: FG30TG) and used in the Oxidative Stability Instrument at 80° C. with the air flow rate of 40 psi. The solubility of different amounts and types of antioxidants used in OSI was checked before and after the application.

For the determination of Oil Stability Indices of crude algal oil (Lot #VY00010309), only oil soluble compounds were used since the compounds which were not soluble in fish oil clearly showed relatively low stability indices. Crude algal oil contained about 1.6 mg/g of mixed natural tocopherols (MNT) prior to use in these experiments whereas fish oil did not contain any antioxidants.

The compounds of formulae (6) and (9) (see Table 14 below), compound of formula (4) (see Table 15 below) and compound of formula (8) (see Table 16 below) were used at different times in the Oxidative Stability instruments under similar operational conditions used for fish oil evaluations. MNT and the oil without any antioxidants (“Blank”) were always used to compare. Also, the synergistic effect of only oil soluble compounds with ascorbyl palmitate was determined using the OSI values. The polymers generated at the end of the experiment were determined by LC (LC=liquid chromatography).

The solubility of the compounds used in the oxidative stability study are shown in Table 13.

TABLE 13
Solubility of compounds of formulae (4), (6), (8) and (9) in fish oil

| | | | Solubility in fish | |
|---|---|---|---|---|
| | | | oil | |
| Compound | | Amount | Room | |
| of formula | Appearance | (mg/g) | temp. | 800° C. |
| 9 | Off white | 0.5 | Soluble | Soluble |
| | powder | 2.0 | Soluble | Soluble |
| 6 | Light | 0.5 | Soluble | Soluble |
| | yellow | 2.0 | Soluble | Soluble |
| | powder | | | |
| 4 | Grey | 0.5 | Not | Not |
| | crystals | | soluble | soluble |
| | | 2.0 | Not | Not |
| | | | soluble | soluble |
| 8 | Light | 0.5 | soluble | soluble |
| | yellow | 2.0 | soluble | soluble |
| | slightly | | | |
| | viscous | | | |
| | liquid | | | |

Oil Stability Index for these compounds at 500 and 2000 ppm levels, in comparison with the same amounts of MNT, are shown in Table 14-16. Preliminary OSI results indicate that compound of formula (6) is comparable to the effect of MNT for the same level used (see Table 14).

TABLE 14
Oxidative stability of FG30TG fish
oil with compounds of formulae
(6) and (9) (SD = standard deviation)

| | OSI (h) | SD |
|---|---|---|
| Blank (FG30TG) | 4.55 | 0.1 |
| 0.5 mg/g of | 5.75 | 0.1 |
| compound | | |
| of formula (9) | | |
| 2 mg/g of compound | 6.05 | 0.1 |
| of formula (9) | | |
| 0.5 mg/g of compound | 7.05 | 0.1 |
| of formula (6) | | |
| 2 mg/g of compound | 7.30 | 0.1 |
| of formula (6) | | |
| 0.5 mg/g of MNT | 6.88 | 0.2 |
| 2 mg/g of MNT | 7.73 | 0.2 |

TABLE 15
Oxidative stability of FG30TG
fish oil with compound of formula (4)
(SD = standard deviation)

| | OSI (h) | SD |
|---|---|---|
| Blank (FG30TG) | 4.70 | 0.1 |
| 0.5 mg/g of compound | 4.98 | 0.3 |
| of formula (4) | | |
| 2 mg/g of compound | 5.95 | 0.4 |
| of formula (4) | | |
| 0.5 mg/g of MNT | 6.93 | 0.2 |
| 2 mg/g of MNT | 7.93 | 0.1 |

TABLE 16
Oxidative stability of FG30TG
fish oil with compound of formula
(8) (SD = standard deviation)

| | OSI (h) | SD |
|---|---|---|
| Blank (FG30TG) | 4.73 | 0.4 |
| 0.5 mg/g of compound | 5.48 | 0.1 |
| of formula 8 | | |
| 2 mg/g of compound | 5.96 | 0.1 |
| of formula 8 | | |
| 0.5 mg/g of MNT | 7.15 | 0.3 |
| 2 mg/g of MNT | 8.38 | 0.4 |

The Protection Factors of the corresponding antioxidant compounds in fish oil are shown as a percentage in Tables 17-19.

The Protection Factors (PF) for each compound in oil were calculated in percentage as:

$$PF ( \% ) = \frac{100 \% \times ( OSI of the sample with compound - OSI of the sample without compound )}{OSI of the sample without compound}$$

TABLE 17
Protection Factors of compounds
of formulae (6) and (9) in
FG30TG fish oil

| | Protection |
|---|---|
| | Factor (%) |
| 0.5 mg/g of compound | 26.37 |
| of formula 9 | |
| 2 mg/g of compound | 32.97 |
| of formula (9) | |
| 0.5 mg/g of compound | 54.95 |
| of formula (6) | |
| 2 mg/g of compound | 60.44 |
| of formula (6) | |
| 0.5 mg/g of MNT | 51.10 |
| 2 mg/g of MNT | 69.78 |

TABLE 18
Protection Factors of compound of
formula (4) in FG30TG fish oil

| | Protection |
|---|---|
| | Factor (%) |
| 0.5 mg/g of compound | 4.74 |
| of formula (4) | |
| 2 mg/g of compound | 25.26 |
| of formula (4) | |
| 0.5 mg/g of MNT | 45.79 |
| 2 mg/g of MNT | 66.84 |

TABLE 19
Protection Factors of compound of
formula (8) in FG30TG fish oil

| | Protection |
|---|---|
| | Factor (%) |
| 0.5 mg/g of compound | 15.42 |
| of formula 8 | |
| 2 mg/g of compound | 25.89 |
| of formula 8 | |
| 0.5 mg/g of MNT | 49.59 |
| 2 mg/g of MNT | 77.06 |

Improvement of the oxidative stability of oil soluble compounds of formulae 6 and 9 when combined with AP is shown in Table 20 whereas Table 21 shows the same synergistic effect of compound of formula (8) with AP.

TABLE 20
Improvement of the effect of the
compounds (6) and of formulae (9)
in FG30TG fish oil using AP
(SD = standard deviation)

| | OSI (h) | SD |
|---|---|---|
| Blank (FG30TG) | 4.63 | 0.0 |
| 2 mg/g of compound | 6.30 | 0.4 |
| of formula (9) | | |
| 2 mg/g of compound | 8.93 | 1.0 |
| of formula | | |
| (9) + 0.5 mg/g of AP | | |
| 2 mg/g of compound | 6.15 | 0.4 |
| of formula (6) | | |
| 2 mg/g of compound | 11.20 | 1.1 |
| of formula | | |
| (6) + 0.5 mg/g of AP | | |
| 2 mg/g of MNT | 8.03 | 0.9 |
| 2 mg/g of MNT + | 15.25 | 1.5 |
| 0.5 mg/g of AP | | |

TABLE 21
Improvement of the effect of compound
of formula (8) in FG30TG fish oil
using a synergistic compound (AP)
(SD = standard deviation)

| | OSI (h) | SD |
|---|---|---|
| Blank (FG30TG) | 2.15 | 0.1 |
| 2 mg/g of compound of | 6.58 | 0.1 |
| formula (8) | | |
| 2 mg/g of compound of | 7.03 | 1.5 |
| formula (8) + | | |
| 0.5 mg/g of AP | | |
| 2 mg/g of MNT | 7.58 | 0.3 |
| 2 mg/g of MNT + | 16.25 | 4.0 |
| 0.5 mg/g of AP | | |

Improvements of the Protection Factors of these oil soluble compounds with AP in fish oil are shown in Tables 22 and 23.

TABLE 22
Improvement of the Protection
Factors of compounds of formulae
(6) and (9) with AP in FG30TG fish oil

| | Protection |
|---|---|
| | Factor |
| | (%) |
| 2 mg/g of compound of formula (9) | 36.1 |
| 2 mg/g of compound of formula (9) + | 92.8 |
| 0.5 mg/g of AP | |
| 2 mg/g of compound of formula (6) | 32.8 |
| 2 mg/g of compound of formula (6) + | 141.9 |
| 0.5 mg/g of AP | |
| 2 mg/g of MNT | 73.3 |
| 2 mg/g of MNT + 0.5 mg/g of AP | 229.4 |

TABLE 23
Improvement of the Protection
Factor of compound of formula
(8) with AP in FG30TG fish oil

| | Protection |
|---|---|
| | Factor |
| | (%) |
| 2 mg/g of compound | 145 |
| of formula (8) | |
| 2 mg/g of compound | 130 |
| of formula | |
| (8) + 0.5 mg/g of AP | |
| 2 mg/g of MNT | 60 |
| 2 mg/g of MNT + 0.5 | 194 |
| mg/g of AP | |

Polymers generated at the end of the stabilization experiment of fish oil with compounds of formulae 6 and 9 and AP are shown in Table 24.

TABLE 24
Reduction of polymers in FG30TG
oil with a compound (AP) synergistic
to compounds of formulae (6) and
(9) (SD = standard deviation)

| | Polymers | |
|---|---|---|
| | (%) | SD |
| Blank (FG30TG) | 43.97 | 3.7 |
| 2 mg/g of compound | 40.34 | 2.0 |
| of formula (9) | | |
| 2 mg/g of compound | 31.06 | 3.2 |
| of formula | | |
| (9) + 0.5 mg/g of AP | | |
| 2 mg/g of compound | 37.37 | 2.6 |
| of formula (6) | | |
| 2 mg/g of compound | 25.09 | 4.6 |
| of formula | | |
| (6) + 0.5mg/g of AP | | |
| 2 mg/g of MNT | 33.87 | 1.1 |
| 2 mg/g of MNT + 0.5 | 12.72 | 2.6 |
| mg/g of AP | | |

Tables 25, 26 and 27 show the PV (peroxide value), p-AV (p-anisidine value) and CD (Conjugated dienoic acid %) of the fish oil samples stabilized with compounds of formulae (6), (8) and (9), respectively.

TABLE 25
Variation of PV with compounds of formulae
(6), (8) and (9) in FG30TG

| | | 4 | 6 | 8 | 11 | 13 | 17 |
|---|---|---|---|---|---|---|---|
| | Initial | days | days | days | days | days | days |
| Blank (FG30TG) | 0.9 | 1.6 | 2.2 | 2.7 | 5.6 | 7.8 | 11.9 |
| 2 mg/g of MNT | 0.9 | 1.1 | 1.2 | 1.4 | 1.7 | 1.5 | 2.1 |
| 2 mg/g of | 0.9 | 2.9 | 7.3 | 8.8 | 11.2 | 15 | 19.2 |
| compound of | | | | | | | |
| formula (9) | | | | | | | |
| 2 mg/g of | 0.9 | 4.1 | 7 | 7.9 | 10.2 | 14.9 | 18.6 |
| compound of | | | | | | | |
| formula (6) | | | | | | | |
| 2 mg/g of | 0.9 | 1.6 | 1.8 | 3.1 | 3.7 | 8.7 | 11.5 |
| compound of | | | | | | | |
| formula (8) | | | | | | | |

TABLE 26
Variation of p-AV (p-anisidine value) with compounds of formulae
(6), (8) and (9) in FG30TG

| | | | | | 11 | 13 | 17 |
|---|---|---|---|---|---|---|---|
| | Initial | 4 days | 6 days | 8 days | days | days | days |
| Blank (FG30TG) | 9.9 | 9.8 | 9.9 | 10.5 | 10.9 | 11.2 | 11.9 |
| 2 mg/g of MNT | 9.9 | 10.3 | 9.9 | 10.1 | 10 | 9.8 | 10 |
| 2 mg/g of | 9.9 | 10.5 | 9.9 | 10.3 | 10.5 | 10.5 | 11 |
| compound of | | | | | | | |
| formula (9) | | | | | | | |
| 2 mg/g of | 9.9 | 10.4 | 9.7 | 10.2 | 10.4 | 10.3 | 10.7 |
| compound of | | | | | | | |
| formula (6) | | | | | | | |
| 2 mg/g of | 9.9 | 10.3 | 9.8 | 10 | 10.4 | 10.6 | 11 |
| compound of | | | | | | | |
| formula (8) | | | | | | | |

TABLE 27
Variation of CD (conjugated dienoic acid in %) with compounds of
formulae (6), (8) and (9) in FG30TG

| | | | | | 11 | 13 | 15 |
|---|---|---|---|---|---|---|---|
| | Initial | 4 days | 6 days | 8 days | days | days | days |
| Blank (FG30TG) | 0.7 | 0.7 | 0.6 | 0.7 | 0.7 | 0.8 | 0.7 |
| 2 mg/g of MNT | 0.7 | 0.7 | 0.6 | 0.7 | 0.7 | 0.7 | 0.7 |
| 2 mg/g of | 0.7 | 0.7 | 0.7 | 0.8 | 0.9 | 0.8 | 0.8 |
| compound of | | | | | | | |
| formula (9) | | | | | | | |
| 2 mg/g of | 0.7 | 0.7 | 0.7 | 0.7 | 0.8 | 0.8 | 0.8 |
| compound of | | | | | | | |
| formula (6) | | | | | | | |
| 2 mg/g of | 0.7 | 0.6 | 0.6 | 0.2 | 0.7 | 0.7 | 0.8 |
| compound of | | | | | | | |
| formula (8) | | | | | | | |

Results:

To evaluate the efficacy of the compounds of formulae (4), (6), (8) and (9) in algal oils, only oil soluble compounds were used. Some of these compounds showed very similar pattern of OSI in fish oil. The compounds of formulae (6) and (9) had clearly lower OSI values than those of MNT in algal oil and both compounds seem to have possible prooxidant effect at higher levels (2 mg/g). Although some of these novel compounds did not have better oxidative stabilities than the commonly used MNT, their antioxidant effect can be improved to a considerable extent by combining with ascorbyl palmitate (“AP”) (Tables 20-21). Protection Factors of all these compounds, including MNT, in fish oil could be improved by the addition of AP (Tables 22-23) indicating the possibility of combining AP to all these novel compounds to improve the oxidative stability of matrices containing high amounts of unsaturated fatty acids such as fish oil.

A combination of complex, polymeric compounds generated at the end of the oxidation cascade of unsaturated fatty acids indicate the levels of overall oxidation of the matrix. The generation of such polymers in fish oil containing these novel antioxidant compounds could be reduced considerably when AP was added as a synergistic compound (Table 24).

For the storage stability study oil soluble compounds were used in fish oil at 2 mg/g level only. Compared to the same level of MNT, all compounds showed much higher PVs than those of MNT (Table 25). There was no considerable variation in p-AV and CD (Tables 26-27) during the storage.

All compounds showed antioxidant properties in fish oil at different levels.

The oxidative stability of fish oil with compound of formula (9) is comparable to the antioxidative effect of MNT.

Compound of formula (4) which is not soluble in fish oil clearly had lower OSI values than those of MNT indicating poorer antioxidant activity than MNT.

### Example 13: Antioxidant Activities of Compounds of Formulae (3) and (7) in Fish Oil and Algal Oil

The compounds of formulae (3) and (7) have been tested. The blank oil, i.e. oil without any antioxidant, and oil containing “MNT” (as used also in example 12) have been used here as benchmark. Any compound better in antioxidant activity than the blank oil indicates that it has antioxidant activity. The comparison with MNT gives an indication about the amount of the antioxidant effect, relative to the activity of MNT.

The compounds of formulae (3) and (7) were used in both fish and algal oils to see their antioxidant effect in these oils. Antioxidant effect was determined using mainly the Oil Stability Index (OSI).

A storage stability study was performed to compare the variation of primary oxidation products, the hydroperoxides, generated during oxidation, measured in terms of peroxide value (PV) and the secondary oxidation products which were measured and determined as anisidine reactive substances or p-anisidine value (p-AV) of oil samples containing these compounds.

Oxidative Stability

Two concentration levels were used: 0.5 mg/g (low level) and 2 mg/g (high level), respectively, have been added to 5 g of oil and used in the Oxidative Stability Instrument operated at 80° C. with the continuous air flow rate at ˜40 psi. All samples were run in duplicate. The Protection Factors (PF) for each compound, as percentage, in oil were calculated as,

$$PF ( \% ) = \frac{100 \% \times ( OSI of the sample with compound - OSI of the sample without compound )}{OSI of the sample without compound}$$

Storage Stability

The two different concentrations of compounds of formulae (3) and (7) were used for the storage stability study. Each compound and MNT were added, each individually, to 40 g of fish oil samples in 60 ml amber bottles at 0.5 mg/g and 2 mg/g levels, thoroughly mixed and stored at ambient temperature for 19 days. All sample bottles were stored open to air, away from light. Compounds of formulae (3) and (7) were not readily soluble in oil, so they had to be thoroughly mixed with the oil around 40° C. to dissolve completely. Peroxide values (PV) and p-anisidine values (p-AV) were determined at different times for 19 days.

Results

OSI values of the fish oil samples containing the compounds of formulae (3) and (7), in comparison to the same levels of MNT, are shown in Table 28.

TABLE 28
Oxidative Stability Indices (OSI)
of FG30TG fish oil with
compounds of formulae (3) and
(7) (SD = standard deviation)

| | OSI (h) | SD |
|---|---|---|
| Blank (FG30TG) | 5.00 | 0.1 |
| 0.5 mg/g of MNT | 5.88 | 0.0 |
| 2 mg/g of MNT | 6.53 | 0.5 |
| 0.5 mg/g of compound | 7.13 | 0.1 |
| of formula (3) | | |
| 2 mg/g of compound | 7.48 | 0.7 |
| of formula (3) | | |
| 0.5 mg/g of compound | 6.15 | 0.4 |
| of formula (7) | | |
| 2 mg/g of compound | 7.23 | 0.7 |
| of formula (7) | | |

Based on the Oil Stability Index data, compounds of formulae (3) and (7) have antioxidant properties since all fish oil samples containing these compounds, at both low and high levels, showed higher OSI values than those of the oil without any antioxidant (Blank-FG30TG).

Compound of formula (3) showed slightly higher Oil Stability Indices than those of MNT indicating that compound of formula (3) possesses relatively higher antioxidant properties than MNT at the specified concentration levels. Compound of formula (10) showed comparable antioxidant activity to MNT.

Peroxide values of fish oil samples at low (0.5 mg/g) and high levels (2 mg/g) are shown in Tables 29 and 30, respectively, whereas the p-AV of the same samples at low (0.5 mg/g) and high levels (2 mg/g) are shown in Tables 31 and 32, respectively.

TABLE 29
Peroxide values (PV, meq/kg) of compounds of formulae (3) and
(7) during storage at 25° C. (0.5 mg/g level)

| | Initial | 1 day | 5 days | 8 days | 14 days | 19 days |
|---|---|---|---|---|---|---|
| Blank | 1 | 1.1 | 2.3 | 7.7 | 13.2 | 19.1 |
| 0.5 mg/g of | 1 | 1 | 2.3 | 7 | 13.6 | 22.9 |
| MNT | | | | | | |
| 0.5 mg/g of | 1 | 1 | 2 | 6.4 | 12.8 | 20.9 |
| compound of | | | | | | |
| formula (3) | | | | | | |
| 0.5 mg/g of | 1 | 1 | 2 | 6.4 | 11.8 | 19.5 |
| compound of | | | | | | |
| formula (7) | | | | | | |

TABLE 30
Peroxide values (PV, meq/kg) of compounds of formulae
(3) and (7) during storage of compounds
of formulae (3) and (7) at 25° C. (2 mg/g level)

| | Initial | 1 day | 5 days | 8 days | 14 days | 19 days |
|---|---|---|---|---|---|---|
| Blank | 1 | 1.1 | 2.3 | 7.7 | 13.2 | 19.1 |
| 2 mg/g of | 1 | 0.9 | 1.6 | 2.9 | 6.1 | 18.3 |
| MNT | | | | | | |
| 2 mg/g of | 1 | 1 | 1.9 | 6.4 | 14.1 | 26.8 |
| compound of | | | | | | |
| formula (3) | | | | | | |
| 2 mg/g of | 1 | 1 | 1.9 | 8.2 | 16.4 | 27.3 |
| compound of | | | | | | |
| formula (7) | | | | | | |

TABLE 31
p-Anisidine value (p-AV) of compounds of formulae (3) and (7)
during storage at 25° C. (0.5 mg/g level)

| | Initial | 1 day | 5 days | 8 days | 14 days | 19 days |
|---|---|---|---|---|---|---|
| Blank | 9.4 | 9.5 | 9.4 | 11.3 | 12.7 | 14.3 |
| 0.5 mg/g of | 9.4 | 9.5 | 9.3 | 11.7 | 13 | 15.8 |
| MNT | | | | | | |
| 0.5 mg/g of | 9.4 | 9.5 | 9.5 | 11.2 | 11.4 | 12.8 |
| compound of | | | | | | |
| formula (3) | | | | | | |
| 0.5 mg/g of | 9.4 | 9.5 | 9.8 | 11.1 | 12.4 | 12.9 |
| compound off | | | | | | |
| formula (7) | | | | | | |

TABLE 32
p-Anisidine value (p-AV) of compounds of formulae (3) and (7)
during storage at 25° C. (2 mg/g level)

| | Initial | 1 day | 5 days | 8 days | 14 days | 19 days |
|---|---|---|---|---|---|---|
| Blank | 9.4 | 9.5 | 9.4 | 11.3 | 12.7 | 14.3 |
| 2 mg/g of | 9.4 | 9.4 | 9.2 | 10.2 | 10 | 9.9 |
| MNT | | | | | | |
| 2 mg/g of | 9.4 | 9.4 | 9.3 | 10.8 | 11.2 | 11.8 |
| compound of | | | | | | |
| formula (3) | | | | | | |
| 2 mg/g of | 9.4 | 9.3 | 9.3 | 11.1 | 11.9 | 12.9 |
| compound of | | | | | | |
| formula (7) | | | | | | |

Primary oxidation products (hydroperoxides) generated in the fish oil samples containing compound of formula (3) or compound of formula (7) or MNT, determined as peroxide values (PV), did not show a considerable difference among them at both concentration levels used. Also, there was no considerable difference in the variation of p-anisidine values (p-AV) in all these samples except the sample which did not contain any antioxidants. The sample which did not contain any antioxidant had relatively higher p-AV values than all other samples showing that the compounds of formulae (3) and (7) possess antioxidant properties.

The Oil Stability Indices (OSI) of crude algal oil with levels of 0.5 mg/g and 2 mg/g of compounds of formulae (3) or (7) are shown in Table 33.

TABLE 33
Oxidative stability of crude algal oil
with compounds of formulae
(3) or (7) (SD = standard deviation)

| | OSI (h) | SD |
|---|---|---|
| Blank (Crude algal oil) | 15.00 | 0.6 |
| 0.5 mg/g of MNT | 15.70 | 0.8 |
| 2 mg/g of MNT | 15.70 | 0.0 |
| 0.5 mg/g of compound | 17.05 | 0.2 |
| of formula (3) | | |
| 2 mg/g of compound | 18.63 | 0.8 |
| of formula (3) | | |
| 0.5 mg/g of compound | 16.38 | 1.0 |
| of formula (7) | | |
| 2 mg/g of compound | 18.53 | 0.8 |
| of formula (7) | | |

The compounds of formulae (3) and (7) improved the oxidative stability of crude algal oil when compared with an oil sample which did not contain any of these compounds (Table 33) showing the antioxidant effect of these compounds in algal oil as well.

Tables 34 and 35 show the Protection Factors of the compounds of formulae (3) and (7) in fish oil and crude algal oil, respectively.

TABLE 34
Protection Factors of compounds of
formulae (3) and (7) in fish oil (80° C.)

| | Protection |
|---|---|
| | Factor (%) |
| 0.5 mg/g of MNT | 17.5 |
| 0.5 mg/g of compound | 23 |
| of formula (7) | |
| 0.5mg/g of compound | 42.5 |
| of formula (3) | |
| 2 mg/g of MNT | 30.5 |
| 2 mg/g of compound | 44.5 |
| of formula (7) | |
| 2 mg/g of compound | 49.5 |
| of formula (3) | |

TABLE 35
Protection Factors of compounds
of formulae (3) and (7) in crude
algal oil (80° C.)

| | Protection |
|---|---|
| | Factor (%) |
| 0.5 mg/g of MNT | 4.7 |
| 0.5 mg/g of compound | |
| of formula (7) | 9.2 |
| 0.5 mg/g of compound | |
| of formula (3) | 13.7 |
| 2 mg/g of MNT | 4.8 |
| 2 mg/g of compound | 23.5 |
| of formula (7) | |
| 2 mg/g of compound | 24.2 |
| of formula (3) | |

Results

The compounds of formulae (3) and (7) have antioxidant properties that are comparable in activity with MNT in fish oil.

Compound of formula (3) has slightly higher Oil Stability Indices compared to MNT.

Application of compound of formula (3) also resulted in the lowest level of secondary oxidation products (p-AV) at levels at 0.5 mg/g concentration. There was no considerable difference in PV between compounds of formulae (3), (7) and MNT during storage.

1. Use of a compound of formula (I) as antioxidant in a feed or in a feed ingredient, 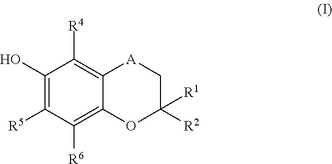
wherein R¹ and R² are independently from each other H or C₁₋₁₁-alkyl or (CH₂)ₙ—OH with n being an integer from 1 to 4, or R¹ and R² represent together a keto group,
A is CHR³ or C(═O), and
wherein R³, R⁴ and R⁶ are independently from each other H or C₁₋₄-alkyl, and wherein R⁵ is H or OH or C₁₋₄-alkyl or C₁₋₄-alkoxy.

2. The use according to claim 1, whereby in compound of formula (I) R¹ and R² are independently from each other H or C₁₋₁₁-alkyl or (CH₂)ₙ—OH with n being an integer from 1 to 4, and R³, R⁴, R⁵ and R⁶ are independently from each other H or C₁₋₄-alkyl or C₁₋₄-alkoxy; preferably whereby in compound of formula (I) R¹ and R² are independently from each other H or C₁₋₄-alkyl or (CH₂)ₙ—OH with n being 1 or 2, R³, R⁴ and R⁶ are independently from each other H or C₁₋₂-alkyl, and R⁵ is H or C₁₋₂-alkyl or C₁₋₂-alkoxy; more preferably whereby in compound of formula (I) R¹ and R² are independently from each other H or methyl or (CH₂)—OH, R³, R⁴ and R⁶ are independently from each other H or methyl, and R⁵ is H or methyl or methoxy; even more preferably with the proviso that one of the substituents R⁴, R⁵ and R⁶ is not methyl.

3. The use according to claim 1, whereby the compound of formula (I) is preferably selected from the group of the compounds of formulae (II) and (III), more preferably from the group of the compounds of formula (IV): 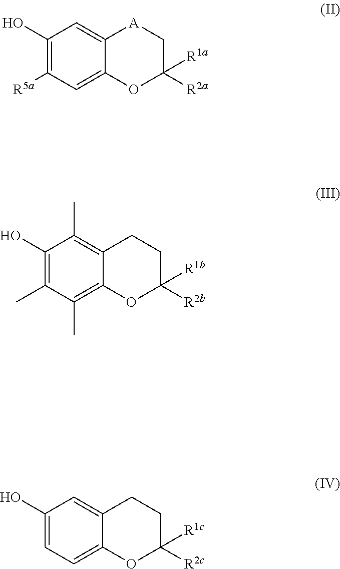
whereby A is CH₂ or C(═O), preferably whereby A is CH₂;
whereby R^{5a} is H or methoxy, preferably whereby R^{5a} is H;
whereby R^{1a} and R^{2a} are independently from each other H, CH₂OH or linear C₁₋₃-alkyl or branched C₄₋₁₁-alkyl or R^{1a} and R^{2a} represent together a keto group (i.e. R^{1a} and R^{2a} are together “═O”), preferably whereby R^{1a} and R^{2a} are independently from each other H, methyl, CH₂OH or [CH₂—CH₂—CH₂—CH(CH₃)]ₙCH₃ with m being 1 or 2 or R^{1a} and R^{2a} represent together a keto group;
whereby R^{1b} and R^{2b} are independently from each other CH₂OH or linear C₁₋₃-alkyl or branched C₄₋₁₁-alkyl, preferably whereby one of R^{1b} and R^{2b} is methyl and the other one of R^{1b} and R^{2b} is CH₂OH or linear C₁₋₃-alkyl or branched C₄₋₁₁-alkyl, more preferably whereby one of R^{1b} and R^{2b} is methyl and the other one of R^{1b} and R^{2b} is methyl, CH₂OH or [CH₂—CH₂—CH₂—CH(CH₃)]ₘCH₃ with m being 1 or 2;
whereby R^{1c} and R^{2c} are independently from each other H or linear C₁₋₃-alkyl or branched C₄₋₁₁-alkyl, preferably whereby R^{1c} and R^{2c} are independently from each other H, methyl or [CH₂—CH₂—CH₂—CH(CH₃)]ₙCH₃ with m being 1 or 2.

4. The use according to claim 1, whereby the compound of formula (I) is one of the following compounds of formulae (1) to (11): 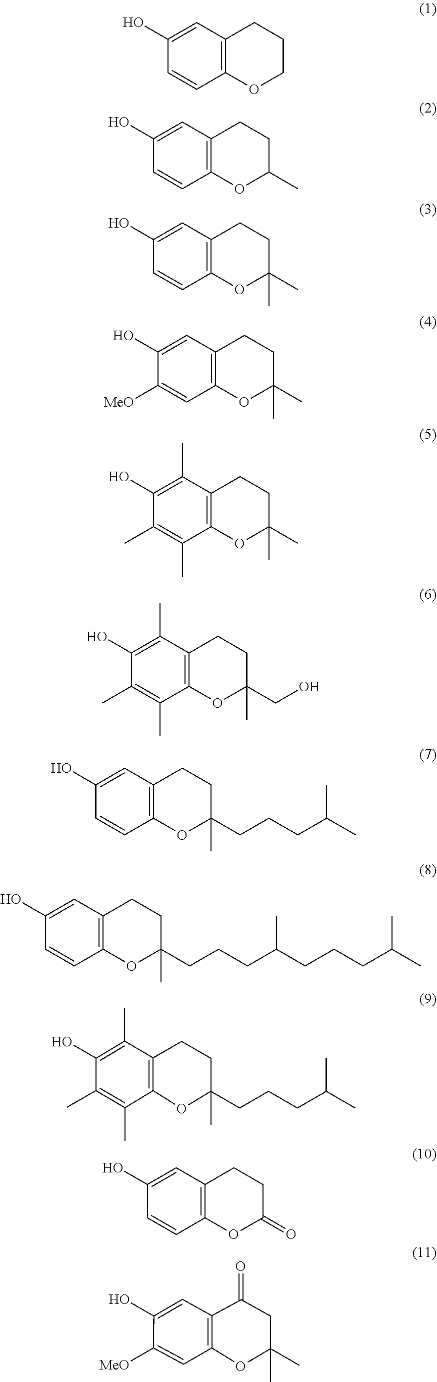
preferably whereby the compound of formula (I) is one of said compounds of formulae (1) to (8).

5. The use according to claim 1, whereby the feed ingredient comprises protein(s) and/or unsaturated fatty acid (derivative)s, preferably whereby the feed ingredient is either poultry meal or fish meal or insect meal or PUFA-containing oil; whereby the PUFA-containing oil is preferably marine oil or microbial oil or fungal oil or algal oil or PUFA-containing plant oil, more preferably whereby the PUFA-containing oil is marine oil or algal oil, even more preferably whereby the PUFA-containing oil is algal oil.

6. Feed ingredient comprising at least one compound of formula (I), 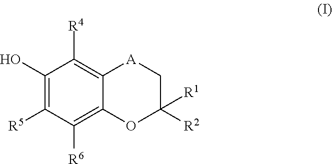
wherein R¹ and R² are independently from each other H or C₁₋₁₁-alkyl or (CH₂)ₙ—OH with n being an integer from 1 to 4, or R¹ and R² represent together a keto group, A is CHR³ or C(═O), and
wherein R³, R⁴ and R⁶ are independently from each other H or C₁₋₄-alkyl, and wherein R⁵ is H or OH or C₁₋₄-alkyl or C₁₋₄-alkoxy.

7. The feed ingredient according to claim 6, whereby the feed ingredient is either poultry meal or fish meal or insect meal or PUFA-containing oil; whereby the PUFA-containing oil is preferably marine oil or microbial oil or fungal oil or algal oil or PUFA-containing plant oil, more preferably whereby the PUFA-containing oil is marine oil or algal oil, even more preferably whereby the PUFA-containing oil is algal oil.

8. The feed ingredient according to claim 6 additionally comprising a mixture of 2-tert-butyl-4-methoxyphenol and 3-tert-butyl-4-methoxyphenol.

9. The feed ingredient according to claim 6 additionally comprising esters of ascorbic acid with linear C₁₂₋₂₀ alkanols, preferably esters of ascorbic acid with linear C₁₄₋₁₈ alkanols, more preferably ascorbyl palmitate.

10. The feed ingredient according to claim 6 additionally comprising alpha-tocopherol and/or gamma-tocopherol.

11. The use according to claim 1, whereby the feed comprises protein(s) and/or unsaturated fatty acid (derivative)s, preferably whereby the feed is feed for aquatic animals, feed for terrestrial animals (preferably feed for poultry, pets or pigs) or feed for insects.

12. Feed for aquatic or terrestrial animals or insects comprising at least one compound of formula (I), 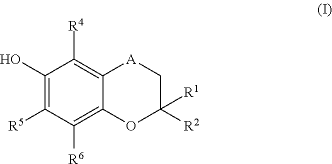
wherein R¹ and R² are independently from each other H or C₁₋₁₁-alkyl or (CH₂)ₙ—OH with n being an integer from 1 to 4, or R¹ and R² represent together a keto group, A is CHR³ or C(═O), and
wherein R³, R⁴ and R⁶ are independently from each other H or C₁₋₄-alkyl, and wherein R⁵ is H or OH or C₁₋₄-alkyl or C₁₋₄-alkoxy.

13. The feed according to claim 12 being pet food, feed for poultry or feed for pigs.

14. 2-(4,8-dimethylnonyl)-2-methyl-chroman-6-ol.
